(19) United States
(12) Patent Application Publication
(10) Pub. No.: US 2026/0166042 A1
(43) Pub. Date: Jun. 18, 2026

(54) METHOD OF MANUFACTURING A PHARMACEUTICAL COMPOSITION COMPRISING NERANDOMILAST

(71) Applicant: Boehringer Ingelheim International GmbH, Ingelheim am Rhein (DE)

(72) Inventors: Hans-Juergen MARTIN, Biberach (DE); Sergej GANN, Waldalgesheim (DE); Thomas HENNES, Neufra (DE); Gerd-Michael MAIER, Biberach (DE); Johanna MILSMANN, Biberach (DE); Tobias RAU, Rottenacker (DE); Odette SCHOENFELD, Ochsenhausen (DE)

(21) Appl. No.: 19/414,692

(22) Filed: Dec. 10, 2025

(30) Foreign Application Priority Data
Dec. 12, 2024 (WO) PCT/EP2024/085899
Oct. 6, 2025 (EP) 25207025.5

(51) Int. Cl. A61K 31/519; A61K 9/20; A61K 9/28

(52) U.S. Cl. CPC A61K 31/519; A61K 9/2013; A61K 9/2018; A61K 9/2054; A61K 9/2095; A61K 9/2813; A61K 9/282; A61K 9/2826; A61K 9/2866; A61K 9/2893

(57) ABSTRACT

The present invention relates to a method of manufacturing a pharmaceutical composition comprising nerandomilast, and the pharmaceutical composition obtained by this method. In the method according to the present invention, the pharmaceutical composition is a tablet comprising nerandomilast, wherein the method comprises the steps of: a) pre-blending nerandomilast and at least one intra-granular excipient, thereby obtaining a pre-blend; b) granulating the pre-blend with a granulation liquid optionally containing a binder using a wet granulation process, thereby obtaining wet granules; c) drying the wet granules, thereby obtaining dry granules; d) blending the dry granules with at least one extra-granular excipient, thereby obtaining a final blend; and e) compressing the final blend using a tablet press equipped with a force feeder, thereby obtaining a tablet; and f) optionally film coating the tablet; wherein the moisture level of the wet granules, the mixing time inside the force feeder and the compression force are adjusted in relation to each other.

### 1 TECHNICAL FIELD

The present invention relates to a method of manufacturing a pharmaceutical composition comprising nerandomilast, and the pharmaceutical composition obtained by this method.

### 2 BACKGROUND ART

Nerandomilast (CAS No. 1423719-30-5) a preferential inhibitor of phorphodiesterase 4B (PDE4B) that is investigated as potential treatment for idiopatic pulmonary fibrosis (IPF) and progressive pulmonary fibrosis (PPF).

In this context, an oral dosage form containing nerandomilast is desired from the viewpoint of improving the patient compliance and allowing patient use at home.

Ideally, an oral dosage form should allow for a reliable drug release. In particular, a tablet allowing for immediate release of nerandomilast is desirable.

Immediate release tablets (also known as conventional release tablets) are tablets in which the release of the active substance is not delayed, for example by means of a special formulation design. One parameter that can be used to assess whether a tablet is an immediate release tablet is the dissolution, expressed as Q value, which refers to the percentage of active ingredient dissolved in a certain medium within a certain time. An immediate release tablet should allow for the dissolution of most of the active substance within a short time, for example within 15, 30 or 45 minutes. This parameter is representative of the drug release performance of the tablet. For tablets for oral administration, it is of particular relevance that this release is tested and confirmed in an environment similar to the gastric environment, i.e., an environment that typically has a pH ranging from 1 to 3.5.

It has been observed that tablets containing nerandomilast tend to show a poor dissolution profile, and it turned out that it is difficult to obtain an immediate release tablet containing this drug by wet granulation.

A method allowing to reliably produce pharmaceutical formulations containing nerandomilast and having an immediate release profile is not known so far.

Accordingly, there is still need for a method of manufacturing a pharmaceutical formulation comprising nerandomilast and having an immediate release profile.

### 3 TECHNICAL PROBLEM

The present invention aims at providing a method of manufacturing a pharmaceutical composition comprising nerandomilast in form of a tablet.

### 4 SUMMARY OF THE INVENTION

The present invention solves the above problem by providing a method of manufacturing a pharmaceutical composition comprising nerandomilast, wherein the pharmaceutical composition is a tablet comprising nerandomilast, wherein the method comprises the steps of:
a) pre-blending nerandomilast and at least one intra-granular excipient, thereby obtaining a pre-blend;
b) granulating the pre-blend with a granulation liquid optionally containing a binder using a wet granulation process, thereby obtaining wet granules;
c) drying the wet granules, thereby obtaining dry granules;
d) blending the dry granules with at least one extra-granular excipient, thereby obtaining a final blend; and
e) compressing the final blend using a tablet press equipped with a force feeder, thereby obtaining a tablet;
wherein the moisture level of the wet granules, the mixing time inside the force feeder and the compression force are adjusted in relation to each other.

Further, the present invention provides a pharmaceutical composition comprising nerandomilast, wherein
the pharmaceutical composition is a tablet comprising nerandomilast and one or more pharmaceutically acceptable excipients, and
wherein the pharmaceutical composition is obtainable by method a of manufacturing a pharmaceutical composition comprising a wet granulation step and a compression step,
wherein the compression step is carried out by using a tablet press equipped with a force feeder, and
wherein the moisture level of the wet granules, the mixing time inside the force feeder and the compression force are adjusted in relation to each other.

The method according to the present invention allows to obtain a pharmaceutical composition that achieves the release of most of the formulated nerandomilast within an acceptable time at a pH similar to the pH of the gastrointestinal environment.

It has been observed that in the production of tablets comprising nerandomilast, certain parameters of the tablet manufacture have an impact on the drug release. In particular, it has been found that when using a wet granulation method, the moisture level of the wet granules is a key factor in allowing for a fast release of the drug. Moreover, the target tensile strength of the tablet, which can be controlled through the compression force, also has an impact on the dissolution behavior. Further, it has been observed that if the system is set in such a way that overmixing and/or overlubrication occur at the compression stage, the dissolution behavior is negatively affected. This problem is solved by the method of the claimed invention by adjusting (i) the moisture level of the wet granules, (ii) the compression force and (iii) the mixing time in the force feeder of the tablet press in relation to each other. The combined control of these three parameters ensures that tablets comprising nerandomilast having an immediate release profile can be reliably obtained for different dosage strengths.

### 5 BRIEF DESCRIPTION OF THE DRAWINGS

FIG. 1. Dissolution profile of formulation (1) at pH 3. FIG. 2. Dissolution profile of formulation (2) at pH 3.

FIG. 3. Dissolution profile of formulations (4) and (1) at pH 3.

FIG. 4. Dissolution profile of formulations (6) and (2) at pH 3.

FIG. 5. Dissolution profile of formulations (4) and (1) at pH 4.5

FIG. 6. Dissolution profile of formulations (6) and (2) at pH 4.5

FIG. 7. Dissolution profile of formulations (4) and (1) at pH 6.8

FIG. 8. Dissolution profile of formulations (6) and (2) at pH 6.8

FIG. 9. Dissolution profile of Comparative Examples 1a-1d, Examples 3a and 3b

FIG. 10. Dissolution profile of Comparative Example 1e, Examples 3c-3g

FIG. 11. Dissolution profile of Examples 3 h-3m

### 6 DETAILED DESCRIPTION OF THE INVENTION

### 6.1 Method of Manufacturing a Pharmaceutical Composition

The present invention provides a method of manufacturing a pharmaceutical composition.

The method according to the present invention may be a method of manufacturing a pharmaceutical composition, wherein the pharmaceutical composition is as defined in any one of the embodiments defined below.

The method comprises the following main manufacturing steps: pre-blending, granulation and drying, final blending, compression, and optionally film-coating.

The method according to the present invention is a method of manufacturing a pharmaceutical composition comprising nerandomilast, wherein the pharmaceutical composition is a tablet comprising nerandomilast, wherein the method comprises the steps of:
a) pre-blending nerandomilast and at least one intra-granular excipient, thereby obtaining a pre-blend;
b) granulating the pre-blend with a granulation liquid using a wet granulation process, thereby obtaining wet granules;
c) drying the wet granules, thereby obtaining dry granules;
d) blending the dry granules with at least one extra-granular excipient, thereby obtaining a final blend; and
e) compressing the final blend using a tablet press equipped with a force feeder, thereby obtaining a tablet;
   wherein the moisture level of the wet granules, the mixing time inside the force feeder and the compression force are adjusted in relation to each other.

In a preferred embodiment, the tablet obtained by the method according to the present invention is an immediate release tablet.

### 6.1.1 Pre-Blending

The method according to the present invention comprises a step (a) of pre-blending nerandomilast and at least one intra-granular excipient, thereby obtaining a pre-blend.

The term “intra-granular excipient” is used herein to indicate those excipients that are introduced in the manufacturing method such that they will be comprised in the granules. Preferably, the step (a) comprises pre-blending nerandomilast with at least one filler. Further preferably, the step (a) may comprise pre-blending nerandomilast with at least one filler and at least one binder. The filler and binder may be as described below for the pharmaceutical composition according to the present invention.

The pre-blend obtained in step (a) may have a particle size distribution D50 of 30 μm or more and 110 μm or less. The particle size may depend on whether coarse-grade or fine-grade fillers are used in the pre-blending step. If coarse-grade fillers are used, the D50 of the pre-blend may be 30 μm or more and 40 μm or less. If fine-grade fillers are used, the D50 may be 80 μm or more and 110 μm or less.

The particle size distribution represents the cumulative undersize distribution and may be determined by laser diffraction according to Ph. Eur. 2.9.31. The value D50 represents the cumulative undersize distribution of 50% and may be referred to as “median particle size”.

When coarse-grade fillers are used, better flowability of the granules may be achieved. Fine-grade fillers may be used, but they may lead to more cohesive pre-blend, thus resulting in poorer feedability. Accordingly, in a preferred embodiment, coarse-grade fillers are used.

The pre-blending step (a) may comprise a step of first pre-blending (pre-blending I), a step of screening and a step of second pre-blending (pre-blending II).

The first and second pre-blending may each independently be carried out in a blender at a mixing speed of 5 rpm or more, preferably 8 rpm or more and 15 rpm or less, preferably 12 rpm or less. Further, the first and second pre-blending may each independently be carried out for 80 rotations or n more, preferably 90 rotations or more, and 120 rotations or less, preferably 110 rotations or less. The blending time may be 8 minutes or more, preferably 9 minutes or more, and 12 minutes or less, preferably 11 minutes or less.

The screening step may be carried out with a screen having a screen size of approximately 1.3 mm.

### 6.1.2 Granulation

The method according to the present invention comprises a step (b) of granulating the pre-blend with a granulation liquid using a wet granulation process.

The granulation liquid used in step (b) optionally contains a binder. Further, the granulation liquid used in step (b) may be water, or may be a mixture of water and one or more excipients, preferably water and one or more binders. The binder(s) used in the step (b) may be as described below for the binder(s) present in the pharmaceutical composition of the present invention.

In the method according to the present invention, a wet granulation process is used. Preferably, a wet granulation method using a twin-screw granulator is used. This method may reduce complexity during scale-up, because all scales can be processed with the same equipment.

The wet granulation process allows for good content uniformity and a reliable immediate release profile even with different particle sizes of the drug.

In the method of the present invention, the pre-blend may be granulated with a granulation liquid in a twin-screw granulator, which may be equipped with double concave conveying screws without kneading block.

In step (b), the moisture level of the wet granules obtained through wet granulation is an important parameter in terms of achieving the immediate release of the tablets according to the present invention.

When the moisture level of the wet granules is above a certain value, poorer dissolution properties are observed. By suitably adjusting the moisture level of the wet granules with certain parameters of the compression step, namely the mixing time in the force feeder and the compression force, a reliable immediate release can be achieved.

The moisture level of the wet granules is expressed as weight percentage with respect to the total weight of the wet granules and is an indication of the amount of water present in the wet granules during step b), that is, during the wet granulation process. It may be determined as the loss on drying (LoD) of the wet granules.

The LoD may be defined as the percentage of weight lost from a sample when dried and is thus indicative of the total amount of water removed by the drying process. The LoD of a sample may be determined according to Ph. Eur. 2.2.32.

During the following drying step, the moisture content of the wet granules will be reduced to the target level, determined as LoD on the stage of the final blend.

The moisture level of the wet granules can be controlled by the process parameters powder and liquid feed rate. For example, the moisture level of the wet granules can be controlled as follows, when the granules are manufactured by dosing the pre-blend using a gravimetric feeder at a constant powder feed rate (PFR): water is used as granulation liquid, which is dosed with a peristaltic pump at a constant liquid feed rate (LFR); these two parameters define the granulation moisture level (ML) which can be calculated using the following equation:

$$ML [ \% ] = \frac{LFR [ \frac{k g}{h} ]}{PFR [ \frac{k g}{h} ] + LFR [ \frac{k g}{h} ]} \times 100$$

The moisture level of the wet granules is preferably less than 25%. When the moisture level of the wet granules is below 25%, the drug release of the tablet cores is such that an immediate release tablet can be reliably obtained. More preferably, the moisture level of the wet granules is 23% or less, more preferably 22.5% or less, even more preferably 21% or less, and even more preferably 20% or less. Preferably, the moisture of the wet granules is 15% or more, more preferably 16% or more, even more preferably 18% or more.

In some embodiments, the moisture level of the wet granules is 16% to 23%, more preferably 18% to 23%, even more preferably 18% to 22.5%, even more preferably 18% to 21% or 18% to 20%.

The moisture level may have an impact on the particle size distribution (PSD) of the dried granules. Generally, a higher granulation moisture level results in a larger particle size.

The D50 of the wet granules may be 100 μm or more and 600 μm or less. The particle size distribution is defined as provided above.

In some embodiments, the moisture level of the wet granules is 18% and the D50 is 100 μm or more and 600 μm or less.

In some embodiments, the moisture level of the wet granules is 21% and the D50 is 300 μm or more and 400 μm or less.

In some embodiments, the moisture level of the wet granules is 22.5% and the D50 is 350 μm or more and 500 μm or less.

Generally, smaller D50 correlate with a faster dissolution. However, when the moisture level of the wet granules is controlled and adjusted in relation to the compression force and the mixing time inside the force feeder, as in the method of the present invention, an immediate release tablet can be obtained even with different particle sizes.

The powder feed rate is preferably 15 kg/h or more, more preferably 20 kg/h or more, and preferably 45 kg/h or less, more preferably 35 kg/h or less. Most preferably, the powder feed rate is 25 kg/h.

The liquid feed rate is 60 g/min or more, preferably 70 g/min or more, more preferably 80 g/min or more, and preferably 160 g/min or less, more preferably 140 g/min or less, even more preferably 120 g/min or less, even more preferably 110 g/min or less. Most preferably, the liquid feed rate is 105 g/min.

The barrel fill level in the context of granulation is the volume occupied by the powder and granules inside the granulator in proportion to the maximum barrel channel void ‘free’ volume.

The barrel fill level (FL) for a granulator equipped with conveying element screws is driven by the powder feed rate, the bulk density of the powder pre-blend, the screw speed of the granulator and the geometry of the granulator according to the following equation:

$$FL [ \% ] = \frac{\frac{PFR [ \frac{k g}{s} ]}{{density}_{powd e r} [ \frac{k g}{{m}^{3}} ]}}{\frac{free {volume}_{barrel} [ {m}^{3} ] \times screw speed [ rps ]}{number of turns}} \times 100$$

The powder feed rate, the pre-blend and hence its bulk density as well as the geometry of the granulator and the screws are fixed. As a consequence, the barrel fill level is controlled mainly by the screw speed.

The barrel fill level is preferably 10% or more, more preferably 13% or more, even more preferably 15% or more, and is preferably 30% or less, more preferably 28% or less, even more preferably 25% or less.

Particle agglomeration is caused by intermixing and shearing of the pre-blend and the granulation liquid. By changing the screw speed, the filling of the powder in the granulator can be adjusted. A lower screw speed is preferable from the viewpoint of achieving a better agglomeration of particles and hence a decrease of ungranulated particle fraction.

Preferably, when using a twin-screw granulation method, the screw speed is 300 rpm or more, more preferably 310 rpm or more, more preferably 330 rpm or more, and even more preferably 330 rpm or more. Further, the screw speed is preferably 420 rpm or less, preferably 400 rpm or less, more preferably 390 rpm or less.

### 6.1.3 Drying

The method according to the present invention further comprises a step (c) of drying the wet granules, thereby obtaining dry granules.

Preferably, the granulation and drying processes are carried out in a continuous system.

The wet granules may be transferred via pneumatic conveying system to a continuous fluid bed dryer. The drying conditions determine the moisture level of the dried granules. Such drying conditions include inlet air flow rate and inlet air temperature.

After drying, the dried granules may be conveyed into a collecting container.

The water content of the dry granules is expressed as loss on drying (LoD). Preferably, the dry granules have a LoD of 2.0% or less. This allows for an improved processability of the granules, as well as improved mechanical strength and drug release profile. Preferably, the dry granules have a LoD of 1.5% or less, more preferably 1.2% or less. Preferably, the dry granules have a LoD of 0.5% or more, more preferably 0.7% or more. Most preferably, the LoD of the dry granules is 0.7% to 1.28. The LoD may be defined as provided above.

The LoD may have an impact on the disintegration time. Higher LoD may correlate to an increase in disintegration time. When the LoD is within the above ranges, an optimal disintegration time of the tablet can be achieved.

Further, the LoD may have an impact on the dissolution of the tablet. Higher LoD may correlate to a decrease in dissolution. When the LoD is within the above ranges, a faster dissolution of the tablet can be achieved.

The target LoD may be achieved by controlling drying parameters such as carousel speed, inlet air flow rate and inlet air temperature. For example, the inlet flow rate may be adjusted depending on the moisture level of the wet granules, whereby lower inlet flow rates may be used for lower moisture levels, and higher inlet flow rates may be used for higher moisture levels.

The inlet air temperature is preferably 70° C. or more, more preferably 75° C. or more, and is preferably 150° C. or less, more preferably 120° C. or less.

The inlet air flow rate is preferably 200 m³/h or more, preferably 300 m³/h or more, and preferably 600 m³/h, more preferably 500 m³/h or less.

The carousel rotation speed is 10 rph or more, preferably 15 rpm or more, more preferably 18 rph or more, and preferably 50 rph or less, more preferably 30 rph or less, even more preferably 25 rph or less.

In some embodiments, the moisture level of the wet granules is 16% or more and 19% or less, and the inlet flow rate is 300 m³/h or more and 400 m³/h or less. In some embodiments, the moisture level of the wet granules is 20% or more and 23% or less and the inlet flow rate is 400 m³/h or more, preferably 410 m³/h or more, and 500 m³/h or less.

The water activity of the dry granules is preferably 0.60 or less, more preferably 0.50 or less, and preferably 0.20 or more, more preferably 0.30 or more. The water activity can be defined as a measurement of the amount of free, available water in a sample and it can be derived from the ratio of the vapor pressure of the sample to the vapor pressure of water at the same temperature. The water activity may be determined according to Ph. Eur. 2.9.39.

Preferably the particle size distribution expressed as D50 of the dried granules is 100 μm or more, more preferably 110 μm or more, and even more preferably 120 μm or more. Further, the D50 of the dried granules is preferably 550 μm or less, preferably 500 μm or less, more preferably 450 μm or less. The particle size distribution is defined as provided above.

The bulk density of the granules is preferably 0.30 mg/mL or more, more preferably 0.40 mg/mL or more, even more preferably 0.50 mg/mL or more, and is 1.00 mg/mL or less, more preferably 0.80 mg/mL or less, and even more preferably 0.70 mg/mL or less.

### 6.1.4 Final Blending

The method according to the present invention comprises a step (d) of blending the dry granules with at least one extra-granular excipient, thereby obtaining a final blend.

The term “extra-granular” excipient used herein refers to excipients that are added to formed granules.

Preferably, the at least one extra-granular excipient includes at least one lubricant and at least one disintegrant. The lubricants and disintegrants that may be used in this step are as defined below for the pharmaceutical composition according to the present invention.

The final blending step may comprise a pre-screening of the dried granules, followed by a step of combined screening of the pre-screened granules and blending with the extra-granular phase.

The blending time of the final blending may have an impact on the blend uniformity and thus on the uniformity of the drug content in the pharmaceutical composition.

The blending time is preferably 5 minutes or more, more preferably 8 minutes or more, and even more preferably 10 minutes or more. Further, the blending time is preferably 25 minutes or less, more preferably 20 minutes or less, even more preferably 15 minutes or less.

The screen size of the screening is preferably 0.5 mm or more, more preferably 1.0 mm or more, and 2.0 mm or less, more preferably 1.5 mm or less.

The screening speed is preferably 600 rpm or more and 1000 rpm or less, more preferably 600 rpm or more and 800 rpm or less.

The blending speed is preferably 5 rpm or more, more preferably 8 rpm or more, even more preferably 10 rpm or more, and 20 rpm or less, more preferably 15 rpm or less.

The blending step may be carried out using a diffusion blender. The fill level of the diffusion blender is preferably 40% to 70%, more preferably 50% to 70% from the viewpoint of uniformity of the blend.

The moisture level of the final blend is expressed in terms of loss on drying (LoD). As the granulate constitutes the majority of the final blend, the moisture blend of the final blend is generally comparable to the moisture level of the dried granules. Accordingly, the preferred values for the LOD of the final blend is as described above for the dried granules.

The water activity of the final blend is preferably 0.60 or less, more preferably 0.50 or less, and preferably 0.20 or more, more preferably 0.30 or more. The water activity is defined as provided above.

Preferably the particle size distribution expressed as D50 of the final blend is 100 μm or more, more preferably 110 μm or more, and even more preferably 120 μm or more. Further, the D50 of the final blend is preferably 400 μm or less, preferably 380 μm or less, even more preferably 200 μm or less. The particle size distribution is defined as provided above.

### 6.1.5 Compression

The method according to the present invention includes a step (e) of compressing the final blend using a tablet press equipped with a force feeder, thereby obtaining a tablet.

The final blend may be compressed on a standard rotary tablet press or a single punch tablet press to yield tablet cores.

The tablet press used in the method according to the present invention is equipped with a force feeder. A force feeder, also known as power feeder or simply fill system, is used to allow the filling of die cavities of the tablet press with the granulate.

Typically, the granulate flows through the hopper to the feed frame, then through the chambers of the feed frame, followed by filling of the dies and compaction of the granules or powder.

When the granulate is inserted into the force feeder, the granulate resides for a certain time inside the feed frame, and during this time, mixing typically occurs. It has been observed that the permanence within the chambers of the force feeder and its mixing therein leads to mechanical stress of the granulate, and may lead to overmixing and overlubrication of the granulate, which in turns leads to poorer dissolution properties. That is, overmixing can lead to overlubrication, which may affect disintegration and dissolution of the tablets. Overlubrication can form a hydrophobic layer around the individual particles, which may lead to an increased disintegration time of the tablets and therefore delayed drug release.

The impact of the mixing time inside the feed frame of the force feeder is particularly evident at higher compression forces, or with higher moisture levels of the wet granulates. In this context, the term “mixing time” and “residence time” inside the force feeder are used interchangeably, and they refer to the time spent by the final blend inside the force feeder frame.

Accordingly, the present invention is based on the finding that by controlling these three parameters, i.e., the compression force, the mixing time inside the force feeder, and the moisture level of the wet granules, in relation to each other, overmixing and overlubricating can be reduced (by controlling the mixing time in the force feeder), while the particle size distribution can be controlled (by controlling the moisture level). The additional control of the compression force allows to obtain tablets having a controlled hardness and compaction level, such that the dissolution of the drug can be adequately achieved within the target timeframe. As a result, tablets with an immediate release profile can be reliably obtained.

In a preferred embodiment, the mixing time inside the force feeder is not more than 10 minutes, more preferably not more than 5 minutes. The preferred lower limit of the mixing time inside the force feeder is 0 minutes, that is, the final blend is preferably not subject to mechanical stress inside the force feeder prior to compression.

Force feeders can have different geometries. The force feeder may be a three-chamber force feeder or a cone-shaped force feeder (herein also referred to as cone filling system). A cone-shaped force feeder is a force feeder that typically has a paddle connected to a cone-shaped dispensing structure. Typically, a cone-shaped force feeder has a single chamber and a single paddle. A cone-shaped force feeder is preferably used from the viewpoint of reducing the mechanical stress, i.e., the mixing time inside the force feeder, because the final blend is dispensed into the chamber and distributed by the paddle almost simultaneously, thus reducing overmixing and overlubrication.

The force feeder speed may be 10 rpm or more and 50 rpm or less.

The compression speed may be 50.000 tablets/h or more, 60.000 tablets/h or more, or 80.000 tablets/h or more, and 200.000 tablets/h or less, or 180.000 tablets/h or less. In some embodiments, the compression speed may be 100.000 tablets/h or more and 200.000 tablets/h or less.

The compaction time may be 3.5 ms or more, 4.0 ms or more, or 5.0 ms or more, and 15.0 ms or less, or 13.0 ms or less.

The compression speed and compaction time may be adjusted depending on the dose strength. For example, tablets containing 9 mg of nerandomilast may be compressed with a compression speed of 150.000 tablets/h or more and 300.000 tablets/h or less and a compaction time of 4.0 ms to 7.9 ms, and tablets containing 18 mg of nerandomilast may be compressed with a compression speed of 100.000 tablets/h or more and 200.000 tablets/h or less and a compaction time of 6.0 ms to 11.9 ms.

In some embodiments, the compression force is preferably 3 kN or more, more preferably 4 kN or more, even more preferably 5 kN or more. Further, the compression force is preferably 20 kN or less, preferably 16 kN or less, more preferably 15 kN or less, even more preferably 14 kN or less.

In some embodiments, the compression force is further preferably 8.5 kN or less, preferably 8 kN or less.

In some embodiments, the compression force is preferably above 8.5 kN, preferably 9 kN or more, or 10 kN or more.

### 6.1.6 Relationship Between Moisture Level of the Wet Granules, Compression Force, and Mixing Time Inside the Force Feeder

In the present invention, (i) the moisture level of the wet granules, (ii) the compression force, and (iii) the mixing time inside the force feeder are adjusted in relation to each other. It has been observed that the interplay of these parameters allows to obtain tablets showing an immediate release profile in a reliable way, which is less affected by other parameters such as the drug particle size or the granule particle size, and that can be reliably achieved even with variations in the relative amounts of the excipients.

### (i) Compression Force of 8.5 kN or Less

In a preferred embodiment, the compression force is 8.5 kN or less, the moisture level of the wet granules is 23% or less, and the following relationship between the numerical values of the moisture level of the wet granules and the mixing time inside the force feeder is satisfied:

Mixing time in the force feeder [min]+Moisture level [%]≤32

In other words, the sum of the numerical values of the mixing time in the force feeder and of the moisture level is, in this embodiment, 32 or less. Preferably, the sum of the numerical values of the mixing time in the force feeder and of the moisture level is 31 or less, more preferably 28 or less. Further, the sum of the numerical values of the mixing time in the force feeder and of the moisture level is preferably 10 or more, more preferably 15 or more.

When the above relationship is satisfied, with a compression force of 8.5 kN or less, the moisture level of the wet granules and the mixing time in the feeder are balanced in such a way that overmixing and overlubrication prior to compression can be reduced, thus improving the dissolution profile of the tablet.

In a further preferred embodiment, the compression force is 8 kN or less. The compression force may be preferably 3 kN or more, or 4 kN or more, or 5 kN or more.

The tensile strength of the tablet in this embodiment may be preferably 1.0 MPa or more and 2.0 MPa or less.

In a preferred embodiment, the compression force is 8.5 kN or less, the moisture level of the wet granules is 23% or less, and the following relationship between the numerical values of the compression force, the moisture level of the wet granules and the mixing time inside the force feeder is satisfied:

Compression force [kN]+Mixing time in the force feeder [min]+Moisture level [%]≤38

In other words, in this embodiment, the sum of the numerical values of compression force, mixing time in the force feeder and moisture level is 38 or less. Preferably, the sum is 37.5 or less, and is further preferably 20 or more, more preferably 25 or more.

In a preferred embodiment, the compression force is 8.5 kN or less, preferably 8 kN or less, the moisture level of the wet granules is 16% or more and 23% or less, more preferably 16% or more and 22.5% or less, more preferably 16% or more and 21% or less, even more preferably 18% or more and 20% or less, and the mixing time in the feeder is not more than 10 minutes, preferably not more than 5 minutes. Preferably, the force feeder is a cone fill system.

### (ii) Compression Force Above 8.5 kN

In a further embodiment, the compression force is above 8.5 KN, preferably 9 kN or more or 10 kN or more, the moisture level of the wet granules is 218 or less, and the following relationship between the numerical values of the moisture level of the wet granules and the mixing time inside the force feeder is satisfied:

Mixing time in the force feeder [min]+Moisture level [%]≤30

In other words, with higher compression forces, both the moisture level and the mixing time in the force feeder are preferably reduced, such that a good dissolution profile can be obtained even with a higher compression force, which leads to a higher tensile strength of the tablet. Preferably, in this embodiment, the sum of the numerical value of the mixing time in the force feeder and of the moisture level is 28 or less, more preferably 26 or less. Further, the sum of the numerical value of the mixing time in the force feeder and of the moisture level is preferably 10 or more, more preferably 15 or more.

In a further preferred embodiment, the compression force is above 8.5 kN, preferably 9 kN or more, more preferably 10 kN or more, even more preferably 10.5 kN or more. The compression force may be preferably 20 kN or less, more preferably 15 kN or less.

The tensile strength of the tablet in this embodiment may be preferably more than 2.0 MPa and 3.0 MPa or less.

In a preferred embodiment, the compression force is above 8.5 kN, the moisture level of the wet granules is 21% or less, and the following relationship between the numerical values of the compression force, the moisture level of the wet granules, the mixing time inside the force feeder is satisfied:

Compression force [kN]+Mixing time in the force feeder [min]+Moisture level [%]≤42

In other words, in this embodiment, the sum of the numerical values of compression force, mixing time in the force feeder and moisture level is 42 or less. Preferably, the sum is 41.5 or less, and is further preferably 20 or more, more preferably 25 or more.

In a preferred embodiment, the compression force is above 8.5 kN, preferably 9 kN or more or 10 kN or more, the moisture level of the wet granules is 16% or more and 21% or less, more preferably 18% or more and 21% or less, even more preferably 18% or more and 20% or less, and the mixing time inside the force feeder is not more than 5 minutes. Preferably, the force feeder is a cone fill system.

In a preferred embodiment, the compression force is above 8.5 kN, preferably 9 kN or more or 10 kN or more, the moisture level of the wet granules is 16% or more and 20% or less, more preferably 18% or more and 20% or less, and the mixing time inside the force feeder is not more than 10 minutes. Preferably, the force feeder is a cone fill system.

### (iii) Preferred Embodiments

In a preferred embodiment, the compression force is 3 kN or more and 15 kN or less, preferably 3 kN or more and 8.5 kN or less; the moisture level of the wet granules is not more than 21%, preferably 16% or more and 21% or less, more preferably 18% or more and 21% or less, even more preferably 18% or more and 20% or less; and the force feeder is a cone-shaped force feeder.

In a preferred embodiment, the compression force is 3 kN or more and 15 kN or less, preferably 3 kN or more and 8.5 kN or less; the moisture level of the wet granules is not more than 21%, preferably 16% or more and 21% or less, more preferably 18% or more and 21% or less, even more preferably 18% or more and 20% or less; and the mixing time inside the force feeder is not more than 5 minutes.

In a preferred embodiment, the compression force is 3 kN or more and 15 kN or less, preferably 3 kN or more and 8.5 kN or less; the moisture level of the wet granules is not more than 21%, preferably 16% or more and 21% or less, more preferably 18% or more and 21% or less, even more preferably 18% or more and 20% or less; the mixing time inside the force feeder is not more than 5 minutes, and the force feeder is a cone-shaped force feeder.

### 6.1.7 Film Coating

The step of applying a film coating may comprise preparing a film-coating suspension. The film-coating suspension may be prepared by dispersing a film-coating mixture under stirring in water.

The tablet cores may be coated with the film-coating suspension in a coater to produce film-coated tablets.

The coating step may include a pre-heating step, a film-coating step and a cooling phase. The three phases may be carried out inside the same coating drum.

In the pre-heating step, the tablet cores are pre-heated inside a coating drum.

In the coating step, the tablet cores are coated with a coater inside the coating drum.

The drum rotation speed may be 6 rpm to 12 rpm.

The inlet air flow rate may be 1500 m³/h to 3000 m³/h, preferably 2000 m³/h to 3000 m³/h.

The spray rate may be 140 g/min to 500 g/min, preferably 360 g/min to 500 g/min.

The atomizing air pressure may be 1.0 bar to 2.7 bar.

The outlet air temperature may be 40° C. to 50° C.

In the cooling phase, the film-coated tablets are conditioned back to approximately room temperature.

### 6.2 Pharmaceutical Composition

The present invention relates to a pharmaceutical composition obtainable by a method of manufacturing a pharmaceutical composition comprising a wet granulation step and a compression step, wherein the compression step is carried out by using a tablet press equipped with a force feeder, and wherein the moisture level of the wet granules, the mixing time inside the force feeder and the compression force are adjusted in relation to each other.

Preferably, the relationship between the moisture level of the wet granules, the mixing time inside the force feeder and the compression force is as described above in section 6.1.6, “Relationship between moisture level of the wet granules, compression force, and mixing time inside the force feeder”.

In some embodiments, the pharmaceutical composition according to the present invention is obtainable by the method according to the present invention described above, that is, a method as described above in section 6.1, “Method of manufacturing a pharmaceutical composition”.

The pharmaceutical composition according to the present invention is in the form of a tablet. In some embodiments, the tablet comprises a tablet core. In the absence of a film coating, the term “tablet” may be interchangeable with the term “tablet core” used below. In some embodiments, the tablet further comprises a film coating and thus comprises both a tablet core and a film coating.

In some embodiments, the tablet may be a multilayer tablet. In such cases, the term “tablet layer” may be interchangeable with the term “tablet core” used below. That is, the amounts provided below with respect to the tablet core and the features of the tablet core equally apply to the case in which the tablet core is used as a layer of a multilayer tablet.

In some embodiments below, the amounts are expressed as weight percent with respect to the total weight of the tablet core. To the extent that the tablet does not comprise a coating and thus consists of the tablet core, those amounts also refer to the total weight of the tablet.

### 6.2.1 Tablet Core

### 6.2.1.1 Nerandomilast

The pharmaceutical composition according to the present invention comprises nerandomilast as an active ingredient.

Nerandomilast, also known as BI 1015550, corresponds to (1-(((5R)-2-(4-(5-chloropyrimidin-2-yl) piperidin-1-yl)-5-oxo-6,7-dihydrothieno (3,2-d)pyrimidin-4-yl)amino)cyclobutyl) methanol) and is represented by Formula 1 below.

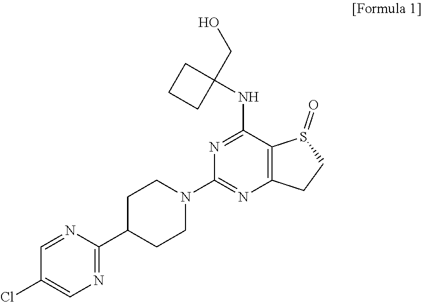 [SMILES: O=[S@@]1CCc2nc(N3CCC(c4ncc(Cl)cn4)CC3)nc(NC3(CO)CCC3)c21]

In the present invention, nerandomilast may be present in its anhydrous forms or in its dihydrate form represented by Formula 2 below.

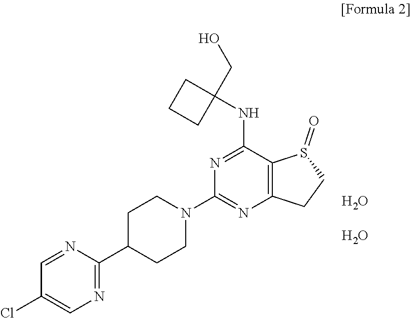

The synthesis and characterization of nerandomilast have been described in WO 2013/026797 A1 and WO 2025/131986 A1.

According to the Biopharmaceutical Classification system (BCS), ICH M9, a drug substance is considered highly soluble when the highest single therapeutic dose strength applied is soluble in 250 mL or less of aqueous media over the pH range of 1-6.8 at 37±1° C. Nerandomilast does not fulfil this criterion over the entire pH range for a dosage strength of 18 mg. At or below pH 3, nerandomilast is very slightly or slightly soluble. Above pH 3, it is practically insoluble.

Six polymorphs (form I-VI) of nerandomilast are known. Preferably, nerandomilast is present in the pharmaceutical composition of the present invention in its Form I or in its Form II, or a mixture thereof.

In some embodiments, the amount of nerandomilast is 4 wt % or more, 5 wt % or more, 6 wt % or more, or 7 wt % or more with respect to the total weight of the tablet core. In some embodiments, the amount of nerandomilast is 15 wt % or less, 10 wt % or less, 9 wt % or less, or 8 wt % or less with respect to the total weight of the tablet core.

The above amount may also be referred to as drug load.

In some embodiments, nerandomilast is present in an amount of 5 mg to 20 mg per tablet. In some embodiments, nerandomilast is present in an amount of 6 mg to 18 mg per tablet. In some embodiments, nerandomilast is present in an amount of 9 to 18 mg per tablet.

In a preferred embodiment, nerandomilast is present in an amount of 6 mg per tablet, 9 mg per tablet, 12 mg per tablet, or 18 mg per tablet.

The above amounts may also be referred to as dosage strength.

In a preferred embodiment, nerandomilast is present in an amount of 6 to 8 wt % with respect to the total weight of the tablet core, and in an amount of 9 mg per tablet or 18 mg per tablet.

### 6.2.1.2 Excipients

### (i) Filler

The pharmaceutical composition according to the present invention preferably contains one or more fillers.

Examples of fillers are inert compounds such as lactose, lactose monohydrate, cellulose microcrystalline, inorganic metal oxides, inorganic phosphate such as dicalcium phosphate, or mannitol, or mixtures thereof.

In some embodiments, the filler includes lactose, lactose monohydrate, cellulose microcrystalline or mixtures thereof. In a preferred embodiment, the filler includes lactose monohydrate, cellulose microcrystalline, or a mixture thereof. In a further preferred embodiment, the filler includes or is a mixture of lactose monohydrate and cellulose microcrystalline.

The excipient(s) used as fillers may be preferably of pharmaceutical grade, and further may be preferably of fine grade or coarse grade. More preferably, the excipient(s) used as fillers are of coarse grade. In a preferred embodiment, the filler includes or is a mixture of coarse-grade lactose monohydrate and coarse-grade cellulose microcrystalline.

In some embodiments, the total amount of filler(s) is 60 wt % or more, 75 wt % or more, or 80 wt % or more with respect to the total weight of the tablet core. In some embodiments, the total amount of filler(s) is 95 wt % or less, 90 wt % or less, or 88 wt % or less with respect to the total weight of the tablet core.

In some embodiments, the filler includes lactose monohydrate in an amount of 50 wt % or more, 60 wt % or more, or 65 wt % or more with respect to the total weight of the tablet core. In some embodiments, the filler includes lactose monohydrate in an amount of 80 wt % or less, 75 wt % or less, or 70 wt % or less with respect to the total weight of the tablet core.

In some embodiments, the filler includes lactose monohydrate in an amount of 50 to 200 mg, or 80 mg to 200 mg, or 80 to 180 mg per tablet.

In some embodiments, the filler includes cellulose microcrystalline in an amount of 15 wt % or more, or 20 wt % or more with respect to the total weight of the tablet core. In some embodiments, the filler includes cellulose microcrystalline in an amount of 30 wt % or less, or 25 wt % or less with respect to the total weight of the tablet core.

In some embodiments, the filler includes cellulose microcrystalline in an amount of 15 mg to 55 mg, or 20 mg to 55 mg per tablet.

In a preferred embodiment, the pharmaceutical composition comprises lactose monohydrate as filler in an amount of 60 wt % or more and 75 wt % or less with respect to the total weight of the tablet core, and cellulose microcrystalline as filler in an amount of 15 wt % or more and 25 wt % or less with respect to the total weight of the tablet core, wherein the total amount of lactose monohydrate and cellulose microcrystalline is 95 wt % or less with respect to the total weight of the tablet core.

In a preferred embodiment, the pharmaceutical composition comprises lactose monohydrate as a filler in an amount of 80 mg to 175 mg per tablet, and cellulose microcrystalline as a filler in an amount of 20 mg to 55 mg per tablet.

In some embodiments, the pharmaceutical composition comprises lactose monohydrate and cellulose microcrystalline as fillers, in a weight ratio of 1.5 to 3.5, preferably 1.9 to 3.3.

### (ii) Binder

The pharmaceutical composition according to the present invention preferably contains one or more binders.

The term “binder” as used herein denotes an excipient which is suitable for binding other components to one another. Examples of binders are hydroxypropylcellulose, powdered cellulose, microcrystalline cellulose, sorbitol, starch, polyvinylpyrrolidone (povidone), copolymers of vinylpyrrolidone with other vinyl derivatives (copovidone), cellulose derivatives, particularly methylhydroxypropylcellulose, e. g. Methocel A 15 LV, and mixtures of these compounds.

In a preferred embodiment, the binder includes or is hydroxypropylcellulose.

In some embodiments, the total amount of binder(s) is 1 or more, 2 wt % or more, 2.5 wt % or more, or 3 wt % or more with respect to the total weight of the tablet core. In some embodiments, the total amount of binder(s) is 8 wt % or less, 5 wt % or less, or 4 wt % or less with respect to the total weight of the tablet core.

In some embodiments, the pharmaceutical composition comprises hydroxypropylcellulose in an amount of 1 to 5 wt %, preferably 2 to 5 wt % with respect to the total weight of the ta blet core.

In some embodiments, the pharmaceutical composition comprises 2 to 10 mg, 2.5 to 8 mg, 3.5 to 8.0 of mg hydroxypropylcellulose.

### (iii) Disintegrant

The pharmaceutical composition according to the present invention preferably contains one or more disintegrants.

Examples of disintegrants are sodium starch glycolate, crosslinked polyvinylpyrrolidone (crospovidone), croscarmellose sodium salt (sodium of cellulose salt carboxymethyl ether, crosslinked), sodium-carboxymethylcellulose, dried maize starch, colloidal anhydrous silica and mixtures thereof.

In a preferred embodiment, the disintegrant includes or is croscarmellose sodium.

In some embodiments, the total amount of disintegrant(s) is 0.5 wt % or more, 1 wt % or more, or 1.5 wt % or more with respect to the total weight of the tablet core. In some embodiments, the total amount of disintegrant(s) is 5 wt % or less, 4 wt % or less, or 3.5 wt % or less with respect to the total weight of the tablet core.

In some embodiments, the disintegrant includes croscarmellose sodium in an amount of 1 wt % to 5 wt %, preferably 1.5 wt % to 4 wt % with respect to the total weight of the tablet core.

In some embodiments, the pharmaceutical composition comprises 1 to 7 mg, preferably 2.5 to 5.5 mg of croscarmellose sodium per tablet.

### (iv) Lubricant

The pharmaceutical composition according to the present invention preferably contains one or more lubricants.

Examples of lubricants are silicon dioxide, talc, stearic acid, sodium stearyl fumarate, magnesium stearate and glycerol tribehenate, or mixtures thereof.

In a preferred embodiment, the lubricant includes or is magnesium stearate.

In some embodiments, the total amount of lubricant(s) is 0.5 wt % or more, 1.0 wt % or more, 1 wt % or more, with respect to the total weight of the tablet core. In some embodiments, the total amount of lubricant(s) is 2 wt % or less, or 1.5 wt % or less with respect to the total weight of the tablet core.

In some embodiments, the lubricant includes magnesium stearate in an amount of 0.5 wt % to 1.5 wt %, preferably 1 wt % to 1.5 wt % with respect to the total weight of the tablet core.

In some embodiments, the pharmaceutical composition comprises 0.5 mg to 5 mg, preferably 1 mg to 3 mg of magnesium stearate per tablet.

### 6.2.1.3 Exemplary Tablet Core Formulations

In an exemplary embodiment, the tablet core comprises nerandomilast and the following excipients:
(i) Lactose monohydrate as a filler;
(ii) Cellulose microcrystalline as a filler;
(iii) Hydroxypropylcellulose as a binder;
(iv) Croscarmellose sodium as a disintegrant;
(v) Magnesium stearate as a lubricant.

In an exemplary embodiment, the tablet core consists of nerandomilast and the above excipient blend.

When a coating is present, the tablet core may represent 90 wt % or more, 92 wt % or more, 95 wt % or more, or 96 wt % or more of the total weight of the tablet.

The total weight of the tablet core may be 80 mg to 300 mg, 100 mg to 300 mg, preferably 120 mg to 260 mg.

In an exemplary embodiment, the tablet core comprises or consists of:
(i) 6 to 8 wt % nerandomilast,
(ii) 75 to 95 wt % filler(s), optionally corresponding to 60 wt % to 70 wt % of a first filler and 15 wt % to 25 wt % of a second filler,
(iii) 2 to 5 wt % binder(s),
(iv) 1 to 4 wt % disintegrant(s),
(v) 0.5 to 1.5 wt % lubricant(s),
   wherein the above wt % are with respect to the total weight of the tablet core.

In an exemplary embodiment, the tablet core comprises or consists of:
(i) 6 to 8 wt % nerandomilast,
(ii) 60 to 70 wt % lactose monohydrate,
(iii) 15 to 25 wt % cellulose microcrystalline,
(iv) 2 to 5 wt % hydroxypropylcellulose,
(v) 1 to 4 wt % croscarmellose sodium,
(vi) 0.5 to 1.5 wt % magnesium stearate,
   wherein the above wt % are with respect to the total weight of the tablet core.

In an exemplary embodiment, the tablet core comprises or consists of:
(i) 7 wt % nerandomilast,
(ii) 67 wt % lactose monohydrate,
(iii) 20 wt % cellulose microcrystalline,
(iv) 3 wt % hydroxypropylcellulose,
(v) 2 wt % croscarmellose sodium,
(vi) 1 wt % magnesium stearate,
   wherein the above wt % are with respect to the total weight of the tablet core.

In an exemplary embodiment, the tablet core comprises or consists of:
(i) 6 to 18 mg nerandomilast,
(ii) 50 to 200 mg lactose monohydrate,
(iii) 15 to 60 mg cellulose microcrystalline,
(iv) 2 to 10 mg hydroxypropylcellulose,
(v) 1 to 7 mg croscarmellose sodium,
(vi) 0.5 to 5 mg magnesium stearate.

### 6.2.2 Tablet Coating Formulation

In some embodiments, the pharmaceutical composition is in the form of a tablet and comprises a tablet core and a coating. The coating is preferably a film-coating.

In some embodiments, the pharmaceutical composition is in the form of a film-coated tablet and consists of a tablet core and a film-coating.

In some embodiments, the pharmaceutical composition is in the form of a film-coated tablet including a tablet core and a film-coating, whereby the film-coating is directly in contact with the tablet core.

In some embodiments, an additional layer may be present between the tablet core and the film-coating coating. In some embodiments, the tablet is a multilayer tablet comprising the tablet core described above as a layer, and the multilayer tablet is coated with a film-coating.

The coating may represent 10 wt % or less, 8 wt % or less, 5 wt % or less, or 4 wt % or less of the total weight of the tablet.

The total weight of the coating may be 2 mg to 10 mg, preferably 3 mg to 8 mg.

The coating preferably covers the entire surface of the tablet core in a continuous manner.

In some embodiments, the coating contains at least one film-forming agent, at least one plasticizer, at least one anti-adherent, at least one pigment, or at least one coloring agent, or a mixture thereof.

In some embodiments, the coating contains at least one film-forming agent, at least one plasticizer, at least one anti-adherent, and at least one pigment or coloring agent.

Examples of film-forming agents include hypromellose (hydroxypropylmethylcellulose, HPMC), methylcellulose, carboxymethylcellulose sodium, polyvinyl alcohol, and mixtures thereof. Preferably, the coating comprises hypromellose, for example hypromellose 2910, as a film-forming agent.

The total amount of film-forming agent may be 1 mg to 3 mg, preferably 1.0 mg to 2.5 mg per tablet. In some embodiments, the coating comprises hypromellose as a film-forming agent in an amount of 1 mg to 3 mg, preferably 1.0 mg to 2.5 mg per tablet.

The total amount of film-forming agent may be 20 wt % or more, preferably 25 wt % or more, more preferably 30 wt % or more with respect to the total weight of the coating. Further, the total amount of film-forming agent may be 50 wt % or less, preferably 45 wt % or less, more preferably 40 wt % or less with respect to the total weight of the coating.

Examples of plasticizers are mannitol, polyethylene glycol, such as PEG 6000 or PEG 8000, also known as Macrogol 6000 (PEG 6000) or Macrogol 8000 (PEG 8000), glycerin, triacetin and sorbitol. Preferably, the plasticizers include mannitol and polyethylene glycol, preferably mannitol and Macrogol 6000 or Macrogol 8000.

The total amount of plasticizer may be 1 mg to 4 mg, preferably 2 mg to 4 mg per tablet. In some embodiments, the coating comprises a mixture of mannitol and polyethylene glycol (preferably Macrogol 6000 or Macrogol 8000) in a total amount of 1 mg to 4 mg, or 2 mg to 4 mg per tablet.

The total amount of plasticizer may be 20 wt % or more, preferably 25 wt % or more, more preferably 30 wt % or more with respect to the total weight of the coating. Further, the total amount of plasticizer may be 50 wt % or less, preferably 45 wt % or less, more preferably 40 wt % or less with respect to the total weight of the coating.

Examples of anti-adherent agents are talc and glyceryl monostearate. In a preferred embodiment, the anti-adherent agent includes or is talc.

The total amount of anti-adherent may be 0.5 mg to 1.5 mg, preferably 0.7 mg to 1.3 mg per tablet. In some embodiments, the coating comprises talc in an amount of 0.5 mg to 1.5 mg, preferably 0.7 mg to 1.3 mg per tablet.

The total amount of anti-adherent may be 10 wt % or more, preferably 15 wt % or more, more preferably 20 wt % or more with respect to the total weight of the coating. Further, the total amount of anti-adherent may be 50 wt % or less, preferably 45 wt % or less, more preferably 40 wt % or less with respect to the total weight of the coating.

Examples of pigments and coloring agents are one or more inorganic metal oxides such as titanium dioxide (E 171) and/or ferric oxide (E 172). Preferred examples of pigments and coloring agents are iron oxides, for example red iron oxide, yellow iron oxide and black iron oxide.

Preferably, the coating comprises hypromellose as a film-forming agent, mannitol as a plasticizer, Macrogol 6000 or Macrogol 8000 as a plasticizer, and talc as anti-adherent.

In some embodiments, the coating further comprises titanium dioxide and one or more iron oxide pigments, for example titanium dioxide and red iron oxide, or titanium dioxide and yellow iron oxide. The total amount of titanium dioxide and iron oxide pigments may be 0.2 mg to 1.5 mg, preferably 0.4 mg to 1.1 mg per tablet.

The total amount of titanium dioxide and iron oxide pigments may be 5 wt % or more, preferably 10 wt % or more, more preferably 12 wt % or more with respect to the total weight of the coating. Further, the total amount of titanium dioxide and iron oxide pigments may be 25 wt % or less, preferably 20 wt % or less with respect to the total weight of the coating.

In some embodiments, the coating comprises one or more iron oxide pigments, for example red iron oxide or yellow iron oxide. The total amount of iron oxide pigments may be 0.01 mg to 0.05 mg, preferably 0.01 mg to 0.03 mg per tablet.

The total amount of iron oxide pigments is preferably 0.01 wt % or more, more preferably 0.05 wt % or more, and preferably 0.5 wt % or less, more preferably 0.3 wt % or less with respect to the total weight of the coating.

In some embodiments, the coating does not comprise titanium dioxide. In other words, in some embodiments, the pigment and coloring agent mixture comprised in the coating consists of iron oxide pigments.

### 6.2.2.1 Exemplary Coating Formulations

In an exemplary embodiment, the coating formulation comprises or consists of:
(i) Hypromellose as a film-forming agent,
(ii) Mannitol as a plasticizer,
(iii) PEG 8000 as a plasticizer,
(iv) Talc as an anti-adherent agent,
(v) One or more iron oxides as pigments.

In an exemplary embodiment, the coating formulation comprises or consists of:
(i) 30 wt % to 40 wt % of film-forming agent(s),
(ii) 20 wt % to 45 wt % of plasticizer(s),
(iii) 15 wt % to 40 wt % of anti-adherent(s),
(iv) 0.01 wt % to 0.5 wt % of pigment(s) and/or coloring agent(s),
   wherein the above wt % refer to the total weight of the coating.

In an exemplary embodiment, the coating formulation comprises or consists of:
(i) 30 wt % to 40 wt % of hypromellose,
(ii) 15 wt % to 25 wt % of mannitol,
(iii) 8 wt % to 16 wt % of PEG 8000,
(iv) 15 wt % to 40 wt % of talc,
(v) 0.01 wt % to 0.5 wt % of iron oxides,
   wherein the above wt % refer to the total weight of the coating.

In an exemplary embodiment, the coating formulation comprises or consists of:
(i) 35 wt % of hypromellose,
(ii) 20 wt % of mannitol,
(iii) 12 wt % of PEG 8000,
(iv) 32.9 wt % of talc,
(v) 0.1 wt % of iron oxides, such as red iron oxide, yellow iron oxide, black iron oxide or a mixture thereof,
   wherein the above wt % refer to the total weight of the coating.

In an exemplary embodiment, the coating formulation comprises or consists of:
(i) 1 to 5 mg hypromellose,
(ii) 0.5 to 3 mg mannitol,
(iii) 0.2 to 2 mg PEG 8000,
(iv) 0.5 to 4.5 mg talc,
(v) 0.001 to 0.02 mg iron oxides.

### 6.2.3 Exemplary Tablet Formulations

In an exemplary embodiment, the tablet comprises a tablet core as defined above in any one of the exemplary embodiments of section 6.2.1.3, “Exemplary tablet core formulations”, and a coating as defined above in any one of the exemplary embodiments of section 6.2.2.1, “Exemplary coating formulations”.

In a preferred embodiment, the pharmaceutical composition is an immediate release tablet comprising or consisting of: (i) a tablet core comprising or consisting of:
nerandomilast, lactose monohydrate, cellulose microcrystalline, hydroxypropylcellulose, croscarmellose sodium, magnesium stearate; and
(ii) a coating comprising or consisting of: hypromellose, mannitol, macrogol 8000, talc, iron oxide, whereby iron oxide is preferably red iron oxide, black iron oxide, and/or yellow iron oxide.

In a preferred embodiment, the pharmaceutical composition is an immediate release tablet consisting of a tablet core and a coating having any one of the formulations (1) to (6) described in the Examples below.

In a preferred embodiment, the pharmaceutical composition is a tablet comprising a tablet core consisting of the following formulation (A):
(i) 9.000 mg nerandomilast
(ii) 85.350 mg lactose monohydrate
(iii) 25.500 mg cellulose microcrystalline
(iv) 3.825 mg hydrocypropylcellulose
(v) 2.550 mg croscarmellose sodium
(vi) 1.275 mg magnesium stearate,
   whereby the total core mass is 127.50 mg.

In a preferred embodiment, the pharmaceutical composition is a tablet comprising a tablet core consisting of the following formulation (B):
(i) 18.000 mg nerandomilast
(ii) 170.700 mg lactose monohydrate
(iii) 51.000 mg cellulose microcrystalline
(iv) 7.650 mg hydroxypropylcellulose
(v) 5.100 mg croscarmellose sodium
(vi) 2.550 magnesium stearate,
   whereby the total core mass is 255.00 mg.

In a preferred embodiment, the pharmaceutical composition is a tablet comprising a tablet core and a film coating, wherein the tablet core consists of the formulation (A) above, and the film coating consists of the following formulation (C):
(i) 1.400 mg hypromellose 2910
(ii) 0.800 mg mannitol
(iii) 0.480 mg Macrogol 6000
(iv) 0.720 mg talc
(v) 0.590 mg titanium dioxide
(vi) 0.010 mg iron oxide, yellow,
   whereby the total mass of the film coat is 4.000 mg and the total mass of the film-coated tablet is 131.50 mg.

In a preferred embodiment, the pharmaceutical composition is a tablet comprising a tablet core and a film coating, wherein the tablet core consists of the formulation (A) above, and the film coating consists of the following formulation (D):
(i) 2.450 mg hypromellose 2910
(ii) 1.400 mg mannitol
(iii) 0.840 mg Macrogol 8000
(iv) 2.303 mg talc
(v) 0.007 mg iron oxide, yellow,
   whereby the total mass of the film coat is 7.000 mg and the total mass of the film-coated tablet is 134.50 mg.

In a preferred embodiment, the pharmaceutical composition is a tablet comprising a tablet core and a film coating, wherein the tablet core consists of the formulation (B) above, and the film coating consists of the following formulation (E):
(i) 2.450 mg Hypromellose 2910
(ii) 1.400 mg mannitol
(iii) 0.840 mg Macrogol 6000
(iv) 1.260 mg talc
(v) 1.033 mg titanium dioxide
(vi) 0.018 mg iron oxide, red,
   whereby the total mass of the film coat is 7.00 mg and the total mass of the film-coated tablet is 262.00 mg.

In a preferred embodiment, the pharmaceutical composition is a tablet comprising a tablet core and a film coating, wherein the tablet core consists of the formulation (B) above, and the film coating consists of the following formulation (F):
(i) 4.200 mg Hypromellose 2910
(ii) 2.400 mg mannitol
(iii) 1.440 mg Macrogol 8000
(iv) 3.947 mg talc
(v) 0.012 mg iron oxide, red
(vi) 0.001 mg iron oxide, black,
   whereby the total mass of the film coat is 12.00 mg and the total mass of the film-coated tablet is 267.00 mg.

### 6.2.4 Characteristics of the Tablet

### (i) Release Profile

The pharmaceutical composition according to the present invention is in the form of a tablet, which is preferably an immediate release tablet.

The term “immediate release” has its conventional meaning in the art. It may refer to a pharmaceutical composition that allows for the release of a certain amount of drug within a certain time in a certain medium, for example, a tablet that allows for the release of 75% of the drug present in the tablet within 45 minutes in an aqueous medium at pH 3.

The dissolution of the tablets may be tested according to the “Dissolution test” according to Ph. Eur. 2.9.3, Apparatus 1 or Apparatus 2. For Apparatus 1, a stirring speed of 50 to 100 rpm may be selected, while for Apparatus 2, a stirring speed of 50 to 75 rpm may be selected. Dissolution in function of pH may be tested over a pH range of 1 to 6.8. The media investigated may be 0.1 M HCl, 0.01 M HCl, 0.001 M HCl, potassium phosphate buffer (pH 3.0), sodium acetate buffer (pH 4.5), potassium phosphate buffer (pH 6.8).

The temperature for the dissolution test may be 37±0.5° C. Volumes of 500, 900 and 1000 mL may be used.

In a preferred embodiment, the tablet has a Q value at 45 minutes of 75% or more, wherein the Q value indicates the percentage of nerandomilast dissolved in 45 minutes in an aqueous medium at pH 3.

In a more preferred embodiment, the tablet has a Q value at 45 minutes of 80% or more, more preferably 85% or more, wherein the Q value indicates the percentage of nerandomilast dissolved in 45 minutes in an aqueous medium at pH 3.

In a preferred embodiment, the tablet has a Q value at 15 minutes of 50% or more, wherein the Q value indicates the percentage of nerandomilast dissolved in 15 minutes in an aqueous medium at pH 3.

In a preferred embodiment, the tablet has a Q value at 30 minutes of 75% or more, preferably 80% or more, wherein the Q value indicates the percentage of nerandomilast dissolved in 30 minutes in an aqueous medium at pH 3.

In a preferred embodiment, the tablet has a Q value at 60 minutes of 80% or more, wherein the Q value indicates the percentage of nerandomilast dissolved in 60 minutes in an aqueous medium at pH 3.

The aqueous medium used for the determination of the above Q values at pH 3 may be an aqueous solution of HCl at a concentration of 0.001 M.

In a preferred embodiment, the tablet has a Q4.5 value at 45 minutes of 40% or more, wherein the 04.5 value indicates the percentage of nerandomilast dissolved in 45 minutes in an aqueous medium at pH 4.5. The aqueous medium at pH 4.5 may be acetate buffer 0.02 M at pH 4.5.

In a preferred embodiment, the tablet has a Q6.8 value at 45 minutes of 35% or more, wherein the Q6.8 value indicates the percentage of nerandomilast dissolved in 45 minutes in an aqueous medium at pH 6.8. The aqueous medium at pH 6.8 may be phosphate buffer 0.05 M at pH 6.8.

Preferably, the Q values as defined above are determined according to the dissolution test according to Ph. Eur., Apparatus 2, with an agitation of 50 rpm, a volume of the aqueous medium of 900 ml, and at a temperature of 37° C., in the aqueous media as defined above.

### (ii) Disintegration

In a preferred embodiment, the tablet has a disintegration time of 15 minutes or less, preferably 10 minutes or less, even more preferably 5 minutes or less, in water according to Ph. Eur. 2.9.1. Further, the disintegration time may be 10 seconds or more, preferably 20 seconds or more in water according to Ph. Eur. 2.9.1.

In some embodiments, the tablet comprises a tablet core and a film-coating, and the above ranges refer to the tablet cores. In some embodiments, the above ranges refer to the film-coated tablet. In some embodiments, the tablet consists of a tablet core and the above ranges refer to the tablet cores.

### (iii) Mechanical Properties

In a preferred embodiment, the tablet has a tensile strength of 3.0 MPa or less, 2.5 MPa or less, preferably 2.0 MPa or less. Further, the tablet has preferably a tensile strength of 0.5 MPa or more, preferably 1.0 MPa or more. In some embodiments, the tablet has a tensile strength of 1.0 MPa or more and 2.0 MPa or less. In some embodiments, the tablet has a tensile strength of more than 2.0 MPa and 3.0 MPa or less. The tensile strength may be determined with a tablet hardness tester. The tensile strength may be controlled by adjusting the compression force accordingly.

For oval tablets, the tensile strength may be determined as described in Powder 238 Technology (2013) 169-175.

Accordingly, the tensile strength may be calculated according to the following formula:

$${σ}_{t} = \frac{2}{3} ( \frac{10 P}{π {D}^{2} ( 2.84 \frac{t}{D} - 0.126 \frac{t}{W} + 3.15 \frac{W}{D} + 0.01 )} )$$

wherein: σₜ is the tensile strength [MPa], P is the fracture load [N], D is the length of the short axis of the tablet (equivalent to disc diameter) [m], L is the length of the long axis of the tablet [m], t is the overall thickness of the tablet [m], and W is the tablet wall height [m].

The term “fracture load” is used interchangeably with “hardness” or “crushing strength” in pharmaceutical tablet testing.

The crushing strength is preferably 30 N or more, more preferably 35 N or more, even more preferably 40 N or more. Further, the crushing strength may be 90 N or less, preferably 85 N or less. The crushing strength may be adjusted depending on the dose strength. For example, tablets containing 9 mg of nerandomilast may have a mean crushing strength of 30 N or more, preferably 35 N or more, and 60 N or less. Tablets containing 18 mg of nerandomilast may have a mean crushing strength of 50 N or more, preferably 60 N or more and 90 N or less. The crushing strength of the tablet may be determined according to Ph. Eur. (2.9.8).

The friability of the tablet core may affect the product appearance and the mechanical strength. The tablet friability may be determined according to Ph. Eur. (2.9.7) and may be 0.15% or less, preferably 0.14% or less.

### (i) Appearance

In a preferred embodiment, the tablet is round or oval.

For example, the tablet may be a round tablet having a diameter of about 3 mm to 8 mm. In a preferred embodiment, the tablet is an oval tablet having a width of about 3 to 6 mm, and a length of about 7 mm to 13 mm.

The size of the tablet may vary depending on the dose strength. For example, in a preferred embodiment, the tablet comprises 9 mg nerandomilast and is an oval tablet having a width of about 3 mm to 5 mm, preferably 4 mm to 5 mm, and a length of about 7 mm to 13 mm, preferably 8 to 11 mm. In a preferred embodiment, the tablet comprises 18 mg nerandomilast and is an oval tablet having a width of about 4 to 6 mm, preferably 5 to 6 mm, and a length of about 7 mm to 13 mm, preferably 9 to 12 mm.

In some embodiments, the tablet is a round tablet having a diameter of about 6 mm or 8 mm, or an oval tablet having a width of about 5.9 mm and a length of about 12 mm, or an oval tablet having a width of about 4.6 mm and a length of about 9.5 mm.

The tablet core thickness is preferably 2 mm or more and 5 mm or less, more preferably 3 mm or more and 5 mm or less, more preferably 3.0 mm or more and 4.0 mm or less, even more preferably 3.1 mm or more and 3.5 mm or less.

### 6.3 Examples

### 6.3.1 List of Tables

Table 1. Composition of formulations (1) to (6) of Example 1
Table 2. Composition of formulations (1) to (6) of Example 1
Table 3. Process parameters of Example 1
Table 4. Dissolution at 45 minutes and disgregation time of formulations (1) and (2) of Example 1
Table 5. Process parameters of Example 3
Table 6. Characterization of Example 3 and Comparative Example 1 (moisture level 22.5%)
Table 7. Characterization of Example 3 and Comparative Example 1 (moisture level 21%)
Table 8. Characterization of Example 3 (moisture level 18%)
Table 9. Relationship between moisture level, compression force and mixing time inside the force feeder in Example 3 (compression force up to 8.5 kN)
Table 10. Relationship between moisture level, compression force and mixing time inside the force feeder in Example 3 (compression force above 8.5 kN)

### 6.3.2 Example 1

Six tablet formulations (formulations (1) to (6)) were prepared, each having the composition according to Table 1. Table 2 reports the composition of the same tablets, wherein the amounts are indicated as weight percentage with respect to the tablet core or to the tablet coating.

TABLE 1

| | | Amount in mg per tablet | | | | | |
|---|---|---|---|---|---|---|---|
| | | Without TiO₂ | | With TiO₂ | | | |
| | | (1) 9 | (2) 18 | (3) 6 | (4) 9 | (5) 12 | (6) 18 |
| | | mg | mg | mg | mg | mg | mg |
| | | Tablet core | | | | | |
| Active | Nerandomilast | 9.000 | 18.000 | 6.000 | 9.000 | 12.000 | 18.000 |
| ingredient | | | | | | | |
| Filler | Lactose monohydrate | 85.350 | 170.700 | 56.900 | 85.350 | 113.800 | 170.700 |
| | Cellulose microcrystalline | 25.500 | 51.000 | 17.000 | 25.500 | 34.000 | 51.000 |
| Binder | Hydroxypropylcellulose | 3.825 | 7.650 | 2.550 | 3.825 | 5.100 | 7.650 |
| Disintegrant | Croscarmellose sodium | 2.550 | 5.100 | 1.700 | 2.550 | 3.400 | 5.100 |
| Lubricant | Magnesium stearate | 1.275 | 2.550 | 0.850 | 1.275 | 1.700 | 2.550 |
| | | Coating | | | | | |
| Film-forming | Hypromellose 2910 | 2.450 | 4.200 | 1.050 | 1.400 | 1.750 | 2.450 |
| agent | | | | | | | |
| Plasticizer | Mannitol | 1.400 | 2.400 | 0.600 | 0.800 | 1.000 | 1.400 |
| | Macrogol 8000 | 0.840 | 1.440 | 0.360 | 0.480 | 0.600 | 0.840 |
| Anti-adherent | Talc | 2.303 | 3.947 | 0.540 | 0.720 | 0.900 | 1.260 |
| Pigment/color- | TiO2 | | | 0.443 | 0.590 | 0.738 | 1.033 |
| ing agent | Red Iron oxide | | 0.012 | 0.008 | | | 0.018 |
| | Black Iron oxide | | 0.001 | | | | |
| | Yellow Iron oxide | 0.007 | | | 0.010 | 0.013 | |
| | Weight of tablet core (mg) | 127.50 | 255.00 | 85.0 | 127.5 | 170.0 | 255.0 |
| | Weight of coating (mg) | 7.00 | 12.00 | 3.00 | 4.00 | 5.00 | 7.00 |
| | Total weight of tablet (mg) | 134.50 | 267.00 | 88.0 | 131.5 | 175.0 | 262.0 |
| | wt % of coating in the | 5.204 | 4.494 | 3.410 | 3.042 | 2.858 | 2.672 |
| | tablet | | | | | | |
| | wt % of tablet core in the | 94.796 | 95.506 | 96.590 | 96.958 | 97.142 | 97.328 |
| | tablet | | | | | | |

TABLE 2

| | | Amount in % of tablet core | | | | | |
|---|---|---|---|---|---|---|---|
| | | Without TiO₂ | | With TiO₂ | | | |
| | | (1) 9 | (2) 18 | (3) 6 | (4) 9 | (5) 12 | (6) 18 |
| | | mg | mg | mg | mg | mg | mg |
| | | Tablet core | | | | | |
| Active | Nerandomilast | 7.06 | 7.06 | 7.06 | 7.06 | 7.06 | 7.06 |
| ingredient | | | | | | | |
| Filler | Lactose | 66.94 | 66.94 | 66.94 | 66.94 | 66.94 | 66.94 |
| | monohydrate | | | | | | |
| | Cellulose | 20.00 | 20.00 | 20.00 | 20.00 | 20.00 | 20.00 |
| | microcrystalline | | | | | | |
| Binder | Hydroxypropylcellulose | 3.00 | 3.00 | 3.00 | 3.00 | 3.00 | 3.00 |
| Disintegrant | Croscarmellose | 2.00 | 2.00 | 2.00 | 2.00 | 2.00 | 2.00 |
| | sodium | | | | | | |
| Lubricant | Magnesium stearate | 1.00 | 1.00 | 1.00 | 1.00 | 1.00 | 1.00 |
| | | Coating | | | | | |
| Film-forming | Hypromellose 2910 | 35.000 | 35.000 | 34.988 | 35.000 | 34.993 | 34.995 |
| agent | | | | | | | |
| Plasticizer | Mannitol | 20.000 | 20.000 | 19.993 | 20.000 | 19.996 | 19.997 |
| | Macrogol 8000 | 12.000 | 12.000 | 11.996 | 12.000 | 11.998 | 11.998 |
| Anti-adherent | Talc | 32.900 | 32.892 | 17.994 | 18.000 | 17.996 | 17.997 |
| Pigment/color- | TiO2 | 0.000 | 0.000 | 14.762 | 14.750 | 14.757 | 14.755 |
| ing agent | Red Iron oxide | 0.000 | 0.100 | 0.267 | 0.000 | 0.000 | 0.257 |
| | Black Iron oxide | 0.000 | 0.008 | 0.000 | 0.000 | 0.000 | 0.000 |
| | Yellow Iron oxide | 0.100 | 0.000 | 0.000 | 0.250 | 0.260 | 0.000 |

The tablets (1) to (6) were prepared by twin screw wet granulation with the parameter settings summarized in Table 3. Specifically, the production steps were (i) pre-blending, (ii) granulation and drying, (iii) final blending, (iv) tableting and (v) film-coating. Water was used as granulation liquid.

TABLE 3

| | | |
|---|---|---|
| Process step | Process parameter | Parameter settings |

Pre-blending

| Pre-blending I | Blend speed | 10 | rpm |
|---|---|---|---|
| | Revolutions | 100 | rotations |
| Screening | Screen size | approx. 1.3 mm | |
| Pre-blending II | Blend speed | 10 | rpm |
| | Revolutions | 100 | rotations |

Granulation and drying

| Granulation | Powder feed rate | 25 | kg/h |
|---|---|---|---|
| | Liquid feed rate | 105 | g/min |
| | Granulation moisture | 20 | |
| | level (%) | | |
| | Screw speed | 330-390 | rpm |
| Drying | Carousel rotation speed | 20 | rph |
| | Inlet air temperature | 75-90° | C. |
| | Inlet air flow rate | 400-470 | m³/h |

Final blending

| Screening of dried | Screen size | approx. 1.3 mm | |
|---|---|---|---|
| granules with | Screening speed | 600-1000 | rpm |
| excipients | | | |
| Final blending | Blend speed | 10 | rpm |
| | Revolutions | 100 | rotations |

Compression

| | | |
|---|---|---|
| Compression | Type of tablet press | Rotary tablet press |
| | Geometry of feeding | Cone-shaped fill system |
| | frame | |

Film-coating

| | | | |
|---|---|---|---|
| Film-coating | Spray rate | 310-500 | g/min |
| | Outlet air temperature | 47-50° | C. |
| | Inlet air volume (flow | 2800-3000 | m³/h |
| | rate) | | |

In the step (i) of pre-blending, lactose monohydrate, nerandomilast, microcrystalline cellulose and hydroxypropylcellulose were pre-blended in a diffusion blender (pre-blending I) and then screened with a screening mill and subsequently blended again in a diffusion blender (pre-blending II) to obtain a pre-blend. Coarse-grade lactose monohydrate and microcrystalline cellulose were used.

In step (ii) of granulation and drying, the pre-blend was continuously granulated with purified water in a twin-screw granulator. The granulation moisture was adjusted to 20%. The wet granules were conveyed into a fluid bed dryer for drying (screw type: without kneading block). The dried granules were conveyed into the collecting container.

In step (iii) of final blending, the dried granules, croscarmellose sodium and magnesium stearate were screened with a suitable screening mill. The screened material was blended in a diffusion blender to obtain the final blend.

In step (iv) of compressing, the final blend was compressed into tablet cores using a standard rotary tablet press. The tablet press was equipped with a cone-shaped filling system in order to minimize the permanence time of the granules in the force feeder and thus limit the mechanical stress of the samples. The residence time of the final blend inside the cone-shaped filling system was regulated as described above by stopping the process within the time limits described above and removing the part of the final bland that remained in the force feeder.

In step (v) of film-coating, a coating dispersion was prepared, by dispersing a film-coating mixture in purified water in a mixing vessel, to obtain a film-coating suspension. The tablet cores obtained in step (iv) were coated with the film-coating suspension in a pan coater to produce nerandomilast film-coated tablets.

For formulation (1), the main compression force was 8.5 kN or less. For formulation (2), the main compression force was between 8.0 kN and 8.7 kN.

### Dissolution Test

The dissolution test at pH 3 was carried out under the following conditions: paddle apparatus (Apparatus 2, Ph. Eur., USP, JP, ChP), agitation 50 rpm, 900 mL HCl, pH 3, 37° C., determination of dissolution by UV spectrophotometry.

The dissolution profile and disintegration time of tablet cores and film-coated tablets of formulations (1) and (2) is reported in Table 4.

TABLE 4

| | | 9 (Formulation (1) | | | 18 (Formulation (2) | | |
|---|---|---|---|---|---|---|---|
| Dose strength [mg] | | 1 | 2 | 3 | 1 | 2 | 3 |
| Batch tablet cores | | | | | | | |
| Main compression | | n.n. | 3.5 | 6.5 | n.n. | 8.0 | 8.5- |
| force [kN] | | | | | | | 8.7 |
| Disintegration [min:s] | Mean | 1:14 | 1:20 | 1:15 | 1:39 | 1:33 | 1:12 |
| | Min | 0:54 | 0:59 | 0:56 | 1:15 | 1:16 | 0:49 |
| | Max | 1:43 | 1:56 | 1:32 | 2:06 | 1:48 | 1:42 |
| Dissolution Q [%] | Mean | 93 | 93 | 93 | 91 | 92 | 89 |
| t = 45 min | | | | | | | |
| Batch film-coated tablets | | | | | | | |
| Disintegration [min:s] | Mean | 1:57 | 2:34 | 2:18 | 2:43 | 2:32 | 1:54 |
| | Min | 1:43 | 1:54 | 2:04 | 2:16 | 2:00 | 1:38 |
| | Max | 2:13 | 3:10 | 2:40 | 3:14 | 2:54 | 2:22 |
| Dissolution Q [%] | Mean | 90 | 91 | 89 | 90 | 90 | 87 |
| t = 45 min | | | | | | | |

As shown in Table 4, both formulations (1) and (2) achieved a Q [%] of 75% or more at 45 minutes and pH 3, and furthermore achieved a disintegration time well under 15 minutes. The presence of a film coating did not affect significantly the release and disintegration profiles of the tablets.

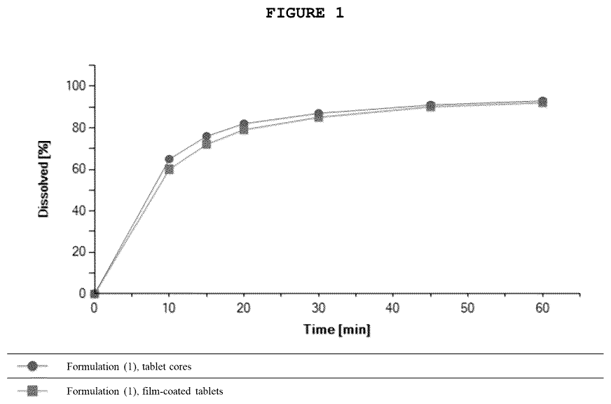

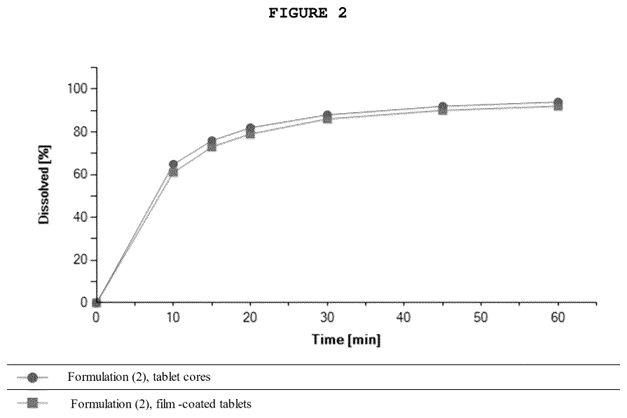

The dissolution profiles at pH 3 of formulations (1) and (2) is furthermore shown in FIG. 1 and FIG. 2, respectively. 6.3.3 Example 2: Dissolution profile at different pH, with and without TiO₂

Tablets having the same formulation as formulations (1), (2), (4) and (6) were prepared. The tablets having the formulation (1) and (2) were prepared with the same method according to Example 1. The tablets having the formulations (4) and (6) were prepared with the same method according to Example 1, except that the liquid feed rate in the wet granulation step was 111 g/min and that a three-chamber fill system was used, whereby the last portion of the final blend was discarded in order to minimize the residence time inside the feeder.

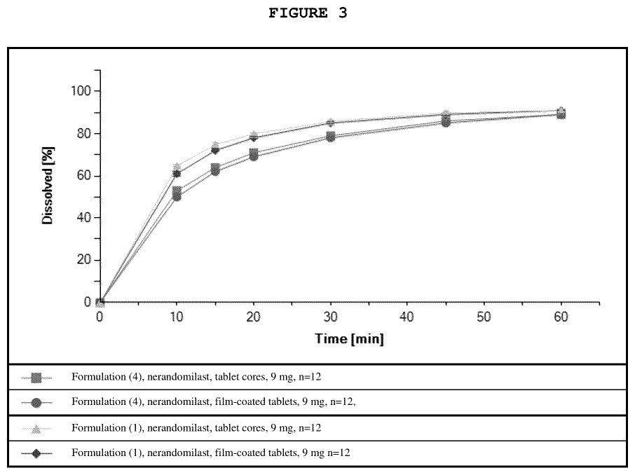

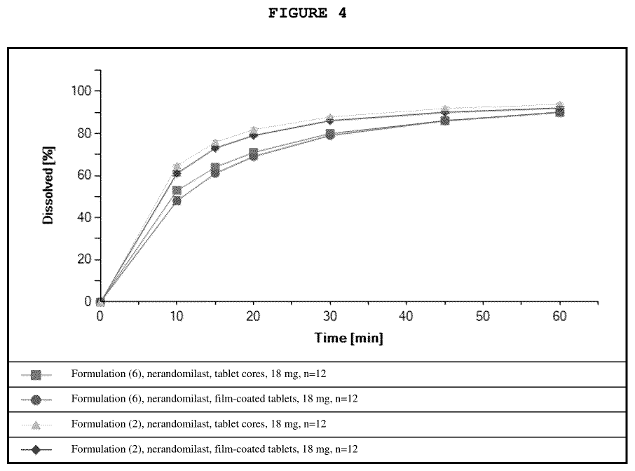

The dissolution profile of formulations (1) and (4) at pH 3 is reported in FIG. 3, whereby the dissolution profile of both the tablet cores and the final film-coated tablets is reported. The dissolution profile of formulations (2) and (6) at pH 3 is reported in FIG. 4, whereby the dissolution profile of both the tablet cores and the final film-coated tablets is reported. All batches achieved a Q value of 75% within 45 minutes. Further, the presence of TiO₂ in the film-coating did not significantly affect the drug release profile. The tablets are thus immediate release tablets.

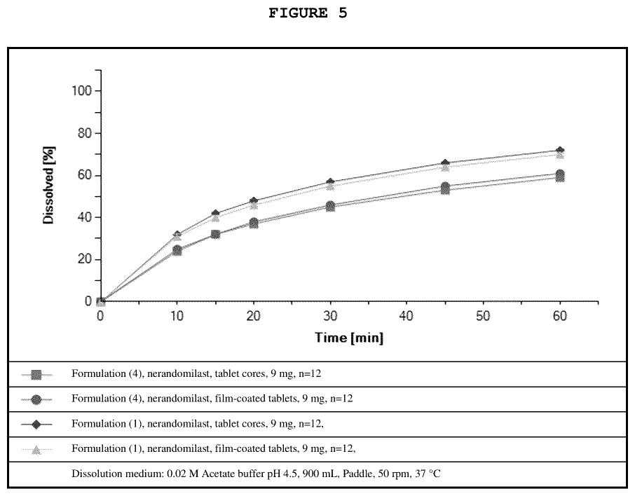

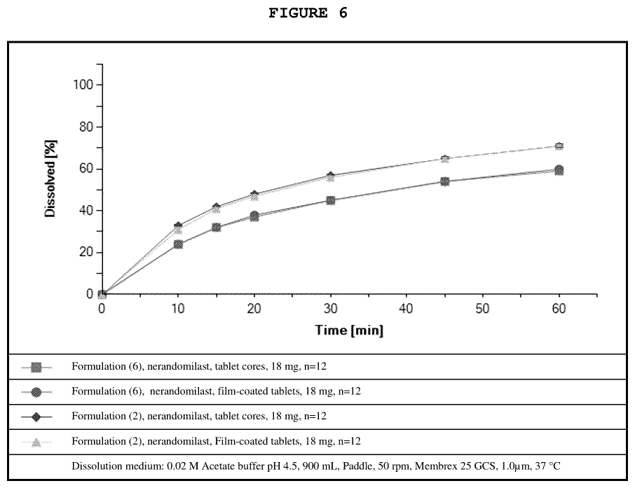

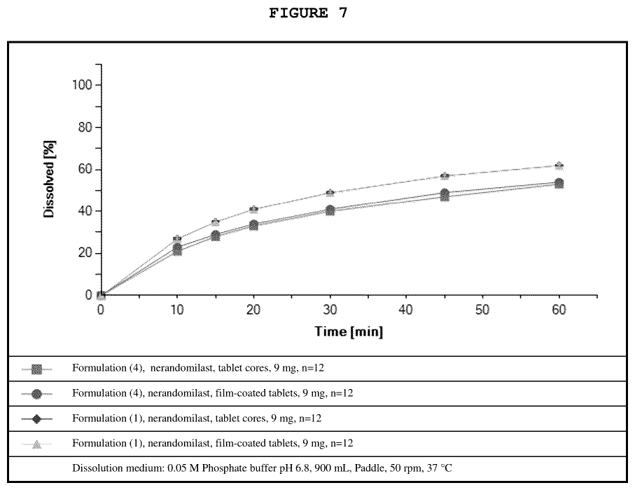

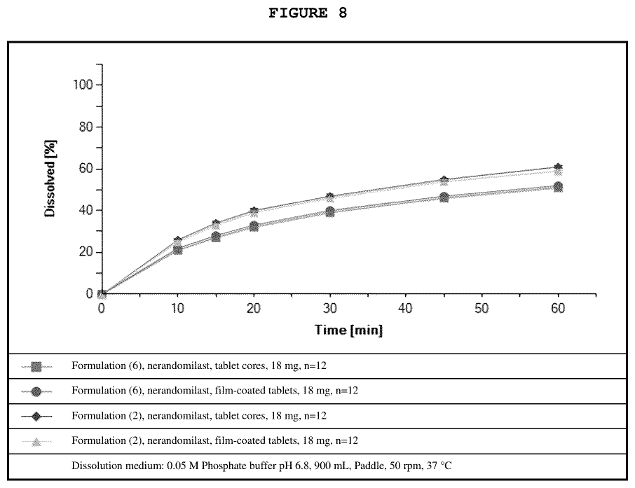

FIGS. 5-8 report the dissolution profiles of formulations (1) and (4) at pH 4.5 (FIG. 5 and FIG. 6), and of formulations (2) and (6) at pH 6.8 (FIG. 7 and FIG. 8), whereby the dissolution profiles of both the tablet cores and the final film-coated tablets are reported. The dissolution test conditions at pH 4.5 were 0.02 M Acetate buffer pH 4.5, 900 mL, Paddle, 50 rpm, 37° C.; the dissolution test conditions at pH 6.8 were 0.05 M Phosphate buffer pH 6.8, 900 mL, Paddle, 50 rpm, 37° C.

### 6.3.4 Example 3 and Comparative Example 1: Relationship Between Moisture Level, Compression Force and Mixing Inside the Force Feeder

Tablet cores having the same composition according to formulation (2) were prepared according to Example 1, except that (i) the moisture level of the wet granules during the granulation process (18%, 21% and 22.5%), (ii) a three-chamber fill system instead of a cone fill system was used, and mechanical stress was induced at the stage prior to compression by keeping the samples in the force feeder for a defined residence time (0 minutes, 5 minutes and 10 minutes), and (iii) tablets were prepared with different compression forces, reflecting different target tensile strengths (1.5 MPa and 2.5 MPa).

The granulation and drying parameters were set as reported in Table 5.

TABLE 5

| Process step | Process parameter | Parameter settings | |
|---|---|---|---|
| Granulation | Powder feed rate | 25 | kg/h |
| | Liquid feed rate | 121 g/min (Granulation | |
| | | moisture 22.5%); | |
| | | 111 g/min (Granulation | |
| | | moisture 21%); | |
| | | 91 g/min (Granulation | |
| | | moisture 18%) | |
| | Screw speed | 310 rpm (Granulation | |
| | | moisture 22.5%); | |
| | | 390 rpm (Granulation | |
| | | moisture 21%); | |
| | | 390 rpm (Granulation | |
| | | moisture 18%) | |
| | Granulation moisture level | 22.5%; | |
| | | 21%, | |
| | | 18% | |
| Drying | Carousel rotation speed | 20 | rph |
| | Inlet air temperature | 80° | C. |

| | |
|---|---|
| Inlet air flow rate | 494 m³/h (Granulation |
| | moisture 22.5%); |
| | 432 m³/h (Granulation |
| | moisture 21%); |
| | 348 m³/h (Granulation |
| | moisture 18%) |

The varied parameters and characteristics of the tablets are reported in Table 6, Table 7 and Table 8.

TABLE 6

| | Granulation moisture level 22.5% | | | | | |
|---|---|---|---|---|---|---|
| PSD Camsizer | D10 [μm] | D50 [μm] | | | | D90 [μm] |
| Granules | 131 | 434 | | | | 1516 |
| Final blend | 47 | 352 | | | | 929 |
| Mixing time in | 0 min | | 5 min | | 10 min | |
| the force | | | | | | |
| feeder | | | | | | |
| | | Comparative | | Comparative | Comparative | Comparative |
| | Example 3a | Example 1a | Example 3b | Example 1b | Example 1c | Example 1d |
| Target tensile | TS 1.5 | TS 2.5 | TS 1.5 | TS 2.5 | TS 1.5 | TS 2.5 |
| strength | | | | | | |
| (MPa) | | | | | | |
| Compression | 5.7 | 8.7 | 5.3 | 9.3 | 6 | 9.5 |
| force [kN] | | | | | | |
| Weight [mg] | 254.65 | 252.66 | 252.43 | 259.53 | 253.64 | 253.69 |
| Thickness | 4.19 | 4 | 4.16 | 4.02 | 4.14 | 3.96 |
| [mm] | | | | | | |
| Crushing | 79.8 | 113.2 | 74.8 | 125.8 | 83 | 117.8 |
| strength [N] | | | | | | |
| Disintegration | 02:36 | 13:31 | 02:58 | 33:35:00 | 09:42 | 27:03:00 |
| time (range) | | | | | | |
| [min:s] | (2:15-3:17) | (11:18- | (2:17-3:52) | (31:21- | (2:32-16:08) | (17:58- |
| | | 15:30) | | 36:43) | | 32:32) |
| Tensile | 1.68 | 2.57 | 1.6 | 2.83 | 1.78 | 2.72 |
| strength | | | | | | |
| [MPa] | | | | | | |
| Solid fraction | 0.83 | 0.88 | 0.83 | 0.9 | 0.84 | 0.89 |
| Dissolution Q | 84 | 63 | 80 | 15 | 27 | 23 |
| [%] at 45 min | | | | | | |

TABLE 7

| | Granulation moisture level 21% | | | | | |
|---|---|---|---|---|---|---|
| PSD Camsizer | D10[μm] | D50 [μm] | | D90 [μm] | | |
| Granules | 74 | 231 | | 1250 | | |
| Final blend | 41 | 185 | | 865 | | |
| Mixing time in | 0 min (unstressed) | | 5 min | | 10 min | |
| the force | | | | | | |
| feeder | | | | | | |
| | | | | | | Comparative |
| | Example 3c | Example 3d | Example 3e | Example 3f | Example 3g | Example 1e |
| Target tensile | TS 1.5 | TS 2.5 | TS 1.5 | TS 2.5 | TS 1.5 | TS 2.5 |
| strength | | | | | | |
| (MPa) | | | | | | |
| Compression | 6.3 | 11.4 | 6.1 | 10.5 | 6.5 | 11.5 |
| force [kN] | | | | | | |
| Weight [mg] | 257.09 | 256.49 | 259.54 | 251.91 | 256.7 | 257.57 |
| Thickness | 4.2 | 3.95 | 4.21 | 3.92 | 4.2 | 3.96 |
| [mm] | | | | | | |
| Crushing | 76 | 119 | 81.4 | 121.6 | 79.4 | 125.6 |
| strength [N] | | | | | | |
| Disintegration | 00:47 | 04:08 | 02:24 | 11:47 | 03:01 | 16:42 |
| time (range) | | | | | | |
| [min:s] | (0:44-0:48) | (3:05-5:10) | (1.55-2:55) | (10:28- | (2:38-3:32) | (15:40- |
| | | | | 12:56) | | 18:00) |
| Tensile | 1.6 | 2.76 | 1.7 | 2.85 | 1.67 | 2.9 |
| strength | | | | | | |
| [MPa] | | | | | | |
| Solid fraction | 0.84 | 0.9 | 0.85 | 0.9 | 0.84 | 0.91 |
| Dissolution Q | 92 | 92 | 91 | 79 | 87 | 68 |
| [%] at 45 min | | | | | | |

TABLE 8

Granulation moisture level 18%

| | | | |
|---|---|---|---|
| PSD Camsizer | D10 [μm] | D50 [μm] | D90 [μm] |
| Granules | 53 | 139 | 693 |
| Final blend | 40 | 126 | 524 |

| | | | |
|---|---|---|---|
| Mixing time in | 0 min (unstressed) | 5 min | 10 min |

the force feeder

| | Example 3h | Example 3i | Example 3j | Example 3k | Example 31 | Example 3m |
|---|---|---|---|---|---|---|
| Target tensile | TS 1.5 | TS 2.5 | TS 1.5 | TS 2.5 | TS 1.5 | TS 2.5 |
| strength (MPa) | | | | | | |
| Compression | 7.5 | 11.1 | 8 | 12.1 | 7.2 | 13.5 |
| force [kN] | | | | | | |
| Weight [mg] | 255.2 | 255.14 | 254.75 | 253.23 | 256.4 | 255.96 |
| Thickness [mm] | 4.1 | 3.95 | 4.05 | 3.89 | 4.1 | 3.9 |
| Crushing | 79.8 | 112.6 | 80.4 | 115.6 | 76.6 | 114.6 |
| strength [N] | | | | | | |
| Disintegration | 00:19 | 00:42 | 00:26 | 01:13 | 00:35 | 02:08 |
| time (range) | (0:18-0:20) | (0:40- | (0:24-0:28) | (1:10-1:20) | (0:31- | (1:52-2:24) |
| [min:s] | | 0:44) | | | 0:37) | |
| Tensile strength | 1.74 | 2.61 | 1.79 | 2.75 | 1.67 | 2.71 |
| [MPa] | | | | | | |
| Solid fraction | 0.86 | 0.9 | 0.87 | 0.91 | 0.86 | 0.92 |
| Dissolution Q | 94 | 95 | 94 | 95 | 95 | 95 |
| [%] at 45 min | | | | | | |

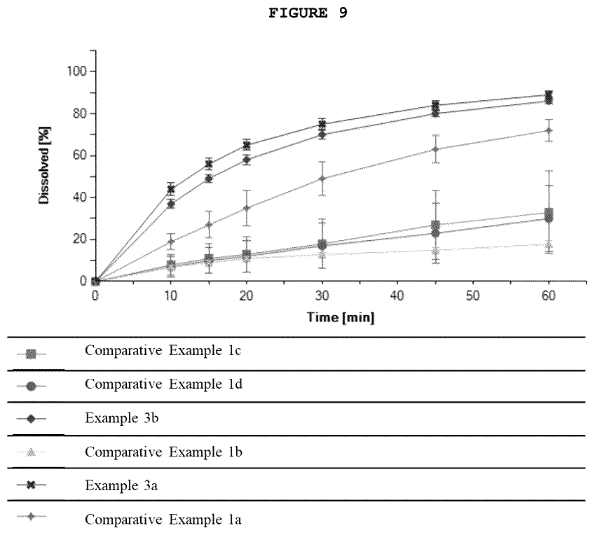

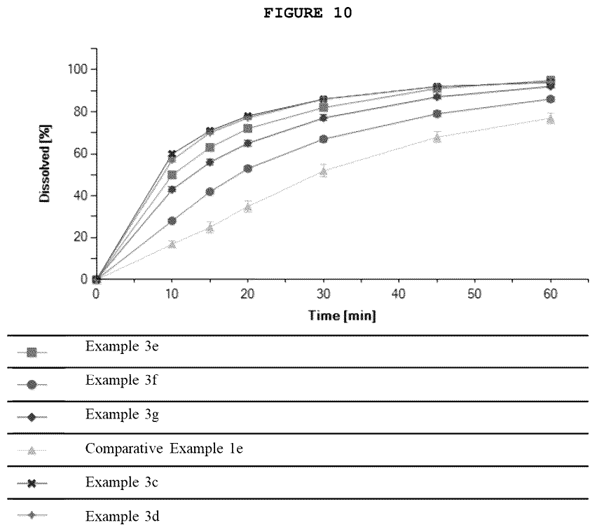

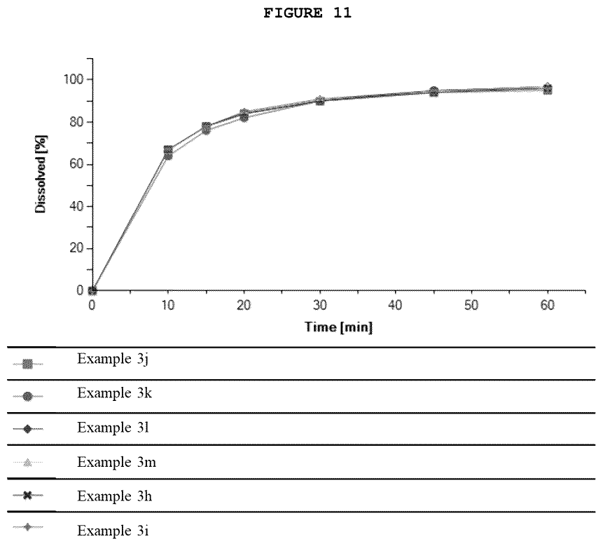

The results of the dissolution test are also reported in FIG. 9 (moisture level 22.5%), FIG. 10 (moisture level 21%) and FIG. 11 (moisture level 18%). The dissolution test was performed in 0.001 M hydrochloric acid, pH 3, 900 mL, paddle, 50 rpm, Musing Membrex 25 GCS, 1.0 μm, at 37° C.

Further, Table 9 and Table 10 report the relationship between the moisture level (ML), the mixing time (MT) inside the force feeder and the compression force (CF) expressed as sum of their numerical values, for samples prepared with a compression force up to 8.5 kN, and above 8.5 kN, respectively.

TABLE 9

| | | | | Target | | |
|---|---|---|---|---|---|---|
| | | | | tensile | | Q [%] at |
| | ML | CF | MT | strength | (ML)/% + | 45 min, |
| | (%) | (kN) | (min) | (Mpa) | (MT)/min | pH 3 |
| Example 3b | 22.5 | 5.3 | 5 | 1.5 | 27.5 | 80 |
| Example 3a | 22.5 | 5.7 | 0 | 1.5 | 22.5 | 84 |
| Comparative | 22.5 | 6 | 10 | 1.5 | 32.5 | 27 |
| Example 1c | | | | | | |
| Example 3c | 21 | 6.3 | 0 | 1.5 | 21 | 92 |
| Example 3e | 21 | 6.1 | 5 | 1.5 | 26 | 91 |
| Example 3g | 21 | 6.5 | 10 | 1.5 | 31 | 87 |
| Example 31 | 18 | 7.2 | 10 | 1.5 | 28 | 95 |
| Example 3h | 18 | 7.5 | 0 | 1.5 | 18 | 94 |
| Example 3j | 18 | 8 | 5 | 1.5 | 23 | 94 |

TABLE 10

| | | | | Target | | |
|---|---|---|---|---|---|---|
| | | | | tensile | | Q [%] at |
| | ML | CF | MT | strength | (ML)/% + | 45 min, |
| | (%) | (kN) | (min) | (Mpa) | (MT)/min | pH 3 |
| Comparative | 22.5 | 8.7 | 0 | 2.5 | 22.5 | 63 |
| Example 1a | | | | | | |
| Comparative | 22.5 | 9.3 | 5 | 2.5 | 27.5 | 15 |
| Example 1b | | | | | | |
| Comparative | 22.5 | 9.5 | 10 | 2.5 | 32.5 | 23 |
| Example 1d | | | | | | |
| Example 3f | 21 | 10.5 | 5 | 2.5 | 26 | 79 |
| Example 3i | 18 | 11.1 | 0 | 2.5 | 18 | 95 |
| Example 3d | 21 | 11.4 | 0 | 2.5 | 21 | 92 |
| Comparative | 21 | 11.5 | 10 | 2.5 | 31 | 68 |
| Example 1e | | | | | | |
| Example 3k | 18 | 12.1 | 5 | 2.5 | 23 | 95 |
| Example 3m | 18 | 13.5 | 10 | 2.5 | 28 | 95 |

It was observed that controlling three specific parameters in combination, namely the target tensile strength of the tablet, which can be controlled through the compression strength, the moisture level during wet granulation, and the mixing time in the force feeder prior to compression, ensures that the resulting tablets reliably show an immediate release profile and achieve a satisfactory dissolution within a short time.

In samples in which the relationship between tensile strength, mixing in the force feeder and moisture level of the wet granulation is not controlled, overmixing/overlubrication may occur. This leads to poorer dissolution properties and to the manufacture of tablets that are not characterized by an immediate release profile.

In particular, it was observed that with a compression force of up to 8.5 kN and a target tensile strength of up to 1.5 MPa, the sum of the numerical values of the moisture level and of the mixing time inside the force feeder should ideally not exceed 32. When the sum is above this threshold, such as in Comparative Example 2c, the three parameters of compression force, moisture level of the wet granules and mixing time inside the force feeder are not appropriately balanced and the dissolution properties are poorer, even though the tablet is prepared with the same method and with the same composition.

With a compression force above 8.5 kN and a higher target tensile strength (up to 2.5 MPa), the dissolution properties are even more affected by the moisture level and mixing time inside the feeder. Accordingly, when targeting higher tensile strengths, the moisture level and mixing time inside the feeder should be even more limited and the sum of their numerical values should not exceed 30.

Generally, for compression forces between 3.0 kN and 15 kN, immediate release tablets could be obtained by controlling the moisture level of the wet granules to not more than 218, and by controlling the mixing time inside the force feeder to not more than 5 minutes.

1. A method of manufacturing a pharmaceutical composition comprising nerandomilast, wherein the pharmaceutical composition is a tablet comprising nerandomilast, wherein the method comprises the steps of:
a) pre-blending nerandomilast and at least one intra-granular excipient, thereby obtaining a pre-blend;
b) granulating the pre-blend with a granulation liquid optionally containing a binder using a wet granulation process, thereby obtaining wet granules;
c) drying the wet granules, thereby obtaining dry granules;
d) blending the dry granules with at least one extra-granular excipient, thereby obtaining a final blend; and
e) compressing the final blend using a tablet press equipped with a force feeder, thereby obtaining a tablet; and
f) optionally film coating the tablet;
wherein the moisture level of the wet granules, the mixing time inside the force feeder and the compression force are adjusted in relation to each other.

2. The method according to claim 1, wherein the tablet is an immediate release tablet.

3. The method according to claim 1, wherein the granulation liquid is water.

4. The method according to claim 1, wherein:
a) the compression force is 8.5 kN or less, the moisture level of the wet granules is 23% or less, and the following relationship between the numerical values of the mixing time in the force feeder and the moisture level of the wet granules is satisfied:
Mixing time in the force feeder [min]+Moisture level [%]≤32; or
b) the compression force is above 8.5 kN, the moisture level of the wet granules is 21% or less, and the following relationship between the numerical values of the moisture level of the wet granules and the mixing time inside the force feeder is satisfied:
Mixing time in the force feeder [min]+Moisture level [%]≤30.

5. The method according to claim 1, wherein
the compression force is 3.0 kN or more and 15.0 kN or less,
the moisture level of the wet granules is 16% or more and 21% or less, and
the mixing time inside the force feeder is not more than 5 minutes.

6. The method according to claim 1, wherein the force feeder is a cone-shaped force feeder.

7. The method according to claim 1, wherein in step b):
the moisture level of the wet granules is 16% or more and 20% or less, and/or
the powder feed rate is 20 Kg/h to 60 Kg/h; and/or
the liquid feed rate is 100 g/min to 125 g/min and/or
the wet granulation process is carried out by a twin-screw wet granulation process, optionally wherein the screw speed is 330 rpm to 390 rpm.

8. A pharmaceutical composition comprising nerandomilast,
wherein the pharmaceutical composition is a tablet comprising nerandomilast and one or more pharmaceutically acceptable excipients, and
wherein the pharmaceutical composition is obtainable by a method of manufacturing a composition comprising a wet granulation step and a compression step,
wherein the compression step is carried out by using a tablet press equipped with a force feeder, and
wherein the moisture level of the wet granules, the mixing time inside the force feeder and the compression force are adjusted in relation to each other.

9. The pharmaceutical composition according to claim 8, wherein the tablet is an immediate release tablet.

10. The pharmaceutical composition according to claim 8, which is obtainable by the method comprising the steps of:
a) pre-blending nerandomilast and at least one intra-granular excipient, thereby obtaining a pre-blend;
b) granulating the pre-blend with a granulation liquid optionally containing a binder using a wet granulation process, thereby obtaining wet granules;
c) drying the wet granules, thereby obtaining dry granules;
d) blending the dry granules with at least one extra-granular excipient, thereby obtaining a final blend; and
e) compressing the final blend using a tablet press equipped with a force feeder, thereby obtaining a tablet; and
f) optionally film coating the tablet;
wherein the moisture level of the wet granules, the mixing time inside the force feeder and the compression force are adjusted in relation to each other.

11. The pharmaceutical composition according to claim 8, wherein:
a) the tablet has a Q value at 45 minutes of 75% or more, wherein the Q value indicates the percentage of nerandomilast dissolved in 45 minutes in an aqueous medium at pH 3; and/or
b) the tablet has a tensile strength of 2.0 MPa or less; and/or
c) the tablet has a disintegration time of 15 minutes or less in water according to Ph. Eur. 2.9.1.

12. The pharmaceutical composition according to claim 8, comprising 5 mg to 20 mg of nerandomilast, lactose monohydrate and cellulose microcrystalline as fillers, hydroxypropyl cellulose as a binder, croscarmellose sodium as a disintegrant and magnesium stearate as a lubricant.

13. The pharmaceutical composition according to claim 8, comprising a tablet core and a film-coating.

14. The pharmaceutical composition according to claim 13, wherein the tablet core comprises:
(i) 7 wt % nerandomilast,
(ii) 67 wt % lactose monohydrate,
(iii) 20 wt % cellulose microcrystalline,
(iv) 3 wt % hydroxypropylcellulose,
(v) 2 wt % croscarmellose sodium, and
(vi) 1 wt % magnesium stearate,
wherein the above wt % are with respect to the total weight of the tablet core; and
wherein the film-coating comprises:
(i) 35 wt % of hypromellose,
(ii) 20 wt % of mannitol,
(iii) 12 wt % of Macrogol 8000,
(iv) 32.9 wt % of talc, and
(v) 0.1 wt % of iron oxides,
wherein the above wt % refer to the total weight of the film-coating.

15. The pharmaceutical composition according to claim 8, wherein the tablet comprises a tablet core and a film-coating, and wherein:
a) the tablet core consists of the following formulation (A), and
(I) the film-coating consists of the following formulation (C) and the total mass of the film-coated tablet is 131.50 mg, or
(II) the film-coating consists of the following formulation (D) and the total mass of the film-coated tablet is 134.50 mg; or
b) the tablet core consists of the following formulation (B) and
(I) the film-coating consists of the following formulation (E) and the total mass of the film-coated tablet is 262.00 mg, or
(II) the film-coating consists of the following formulation (F) and the total mass of the film-coated tablet is 267.00 mg;
Formulation (A):
(i) 9.000 mg nerandomilast,
(ii) 85.350 mg lactose monohydrate,
(iii) 25.500 mg cellulose microcrystalline,
(iv) 3.825 mg hydrocypropylcellulose,
(v) 2.550 mg croscarmellose sodium, and
(vi) 1.275 mg magnesium stearate,
whereby the total core mass is 127.50 mg;
Formulation (B):
(i) 18.000 mg nerandomilast,
(ii) 170.700 mg lactose monohydrate,
(iii) 51.000 mg cellulose microcrystalline,
(iv) 7.650 mg hydroxypropylcellulose,
(v) 5.100 mg croscarmellose sodium, and
(vi) 2.550 magnesium stearate,
whereby the total core mass is 255.00 mg;
Formulation (C):
(i) 1.400 mg hypromellose 2910,
(ii) 0.800 mg mannitol,
(iii) 0.480 mg Macrogol 6000,
(iv) 0.720 mg talc,
(v) 0.590 mg titanium dioxide, and
(vi) 0.010 mg iron oxide, yellow,
whereby the total mass of the film coat is 4.000 mg;
Formulation (D):
(i) 2.450 mg hypromellose 2910,
(ii) 1.400 mg mannitol,
(iii) 0.840 mg Macrogol 8000,
(iv) 2.303 mg talc, and
(v) 0.007 mg iron oxide, yellow,
whereby the total mass of the film coat is 7.000 mg;
Formulation (E):
(i) 2.450 mg Hypromellose 2910,
(ii) 1.400 mg mannitol,
(iii) 0.840 mg Macrogol 6000,
(iv) 1.260 mg talc,
(v) 1.033 mg titanium dioxide, and
(vi) 0.018 mg iron oxide, red,
whereby the total mass of the film coat is 7.00 mg;
Formulation (F):
(i) 4.200 mg Hypromellose 2910,
(ii) 2.400 mg mannitol,
(iii) 1.440 mg Macrogol 8000,
(iv) 3.947 mg talc,
(v) 0.012 mg iron oxide, red, and
(vi) 0.001 mg iron oxide, black,
whereby the total mass of the film coat is 12.00 mg.
